(19) United States
(12) Patent Application Publication
(10) Pub. No.: US 2025/0257353 A1
(43) Pub. Date: Aug. 14, 2025

(54) SIGNAL ACTIVATABLE NUCLEIC ACID COMPLEXES

(71) Applicant: Switch Therapeutics Inc., South San Francisco, CA (US)

(72) Inventors: Si-ping Han, South San Francisco, CA (US); Robert Duff, South San Francisco, CA (US); Lisa Scherer, South San Francisco, CA (US)

(21) Appl. No.: 18/575,487

(22) PCT Filed: Jul. 5, 2022
(86) PCT No.: PCT/US2022/073430

§ 371 Date: Dec. 29, 2023

Related U.S. Application Data
(60) Provisional application No. 63/218,852, filed on Jul. 6, 2021.

(51) Int. Cl. C12N 15/113; A61K 9/00; A61P 25/28

(52) U.S. Cl. CPC C12N 15/113; A61K 9/0085; A61P 25/28; C12N 2310/113; C12N 2310/14; C12N 2310/315; C12N 2310/321; C12N 2310/322; C12N 2310/3231; C12N 2310/351

(57) ABSTRACT

Provided herein include conditionally activatable small interfering RNA (siRNA) complexes, components, compositions, and related methods and systems. The siRNA complex can be conditionally activated upon a complementary binding to an input nucleic acid strand through a sequence in a sensor nucleic acid strand of the nucleic acid complex. The activated nucleic acid complex can release a potent RNAi duplex formed by a core nucleic acid strand and a passenger nucleic acid strand, which can specifically inhibit a target RNA.

### CROSS-REFERENCE TO RELATED APPLICATIONS

This application is a National Stage application of PCT Application PCT/US2022/073430, filed on Jul. 5, 2022, which claims the benefit under 35 U.S.C. § 119 (e) to U.S. Provisional Patent Application No. 63/218,852 filed on Jul. 6, 2021, the content of each of which is incorporated herein by reference in its entirety for all purposes.

### REFERENCE TO SEQUENCE LISTING

The present application is being filed along with a Sequence Listing in electronic format. The Sequence Listing is provided as a file entitled Revised_75EN-329793-US, created Aug. 8, 2024, which is 102,756 bytes in size. The information in the electronic format of the Sequence Listing is incorporated herein by reference in its entirety.

### BACKGROUND

### Field

The present disclosure relates generally to the field of nucleic acid based technology, for example, signal activatable small interfering RNA complexes.

### Description of the Related Art

Despite emerging developments in the field of dynamic nuclei acid nanotechnology and biomolecular computing, there is still a challenge to develop targeted RNAi therapy that can use nuclei acid logic switches to sense RNA transcripts (such as mRNAs and miRNAs), thereby restricting RNA interfering (RNAi) therapy to specific populations of disease-related cells. In particular, there is a need to develop targeted and conditionally activated RNAi therapy with improved drug potency, sensitivity, and stability, low design complexity, and low dosage requirement.

### SUMMARY

Disclosed herein include a nucleic acid complex, comprising: a first nucleic acid strand comprising 20-60 linked nucleosides; a second nucleic acid strand binding to a first region of the first nucleic acid strand to form a first nucleic acid duplex; and a third nucleic acid strand binding to a second region of the first nucleic acid strand to form a second nucleic acid duplex, wherein the third nucleic acid strand comprises a overhang, wherein the overhang is not complementary to the first nucleic acid strand and is capable of binding to an input nucleic acid strand to cause the displacement of the third nucleic acid strand from the first nucleic acid strand. In some embodiments, the first region of the first nucleic acid strand is 3′ of the second region of the first nucleic acid strand, the third nucleic acid strand does not bind to any region of the first nucleic acid strand that is 3′ of the first region of the first nucleic acid strand. In some embodiments, the first region of the first nucleic acid strand comprises a sequence complementary to a target RNA, where the sequence can be, for example, 10-35 nucleosides in length. In some embodiments, the sequence complementary to the target RNA is 10-21 nucleotides in length.

In some embodiments, the second nucleic acid strand binds to 17-22 linked nucleotides in the first region of the first nucleic acid strand to form the first nucleic acid duplex. In some embodiments, the third nucleic acid strand binds to 10-30 linked nucleotides in the second region of the first nucleic acid strand to form the second nucleic acid duplex. In some embodiments, the third nucleic acid strand binds to about 14 linked nucleotides in the second region of the first nucleic acid strand to form the second nucleic acid duplex. In some embodiments, the first nucleic acid duplex, the nucleic acid complex, or both do not comprise a Dicer cleavage site.

In some embodiments, the first region of the first nucleic acid strand is linked to the second region of the first nucleic acid strand via a linker. The linker can, for example, comprise a C3 3-carbon linker, a nucleotide, a modified nucleotide, or a exonuclease cleavage-resistant moiety, or a combination thereof. In some embodiments, the modified nucleotide is a 2′-O-methyl nucleotide or a 2′-F nucleotide. In some embodiments, the 2′-O-methyl nucleotide is 2′-O-methyladenosine, 2′-O-methylguanosine, 2′-O-methyluridine, or 2′-O-methylcytidine. In some embodiments, the 2′-F nucleotide is 2′-F adenosine, 2′-F guanosine, 2′-F uridine, or 2′-F cytidine. In some embodiments, the 5′ terminus of the second nucleic acid strand comprises a blocking moiety. The blocking moiety can, for example, comprise, or is, a fluorophore, an inverted-dT, a tri-ethylene-glycol, a fatty acid, a Cy3, or a combination thereof.

The fluorophore can be attached to the 5′ terminus of the second nucleic strand via a phosphorothioate linkage. In some embodiments, the first nucleic acid strand comprises a 3′ overhang in the first nucleic acid duplex. In some embodiments, the 3′ overhang of the first nucleic acid is one, two, or three nucleotides in length. In some embodiments, the 3′ overhang of the first nucleic acid comprises one or more phosphorothioate internucleoside linkages. In some embodiments, all of the internucleoside linkages in the 3′ overhang of the first nucleic acid are phosphorothioate internucleoside linkages. In some embodiments, the internucleoside linkage(s) between the last two, three or four nucleosides at the 3′ terminus of the first nucleic acid strand is phosphorothioate internucleoside linkage(s). In some embodiments, the first region of the first nucleic acid strand does not comprise phosphorothioate internucleoside linkages except for the internucleoside linkage(s) between the last two or three nucleosides at the 5′ terminus, 3′ terminus, or both. In some embodiments, the first region of the first nucleic acid strand does not comprise phosphorothioate internucleoside linkages except for the internucleoside linkage(s) between the last three nucleosides at the 5′ terminus and the last three nucleosides at 3′ terminus.

In some embodiments, the second region of the first nucleic acid strand does not comprise phosphorothioate internucleoside linkages. In some embodiments, the second nucleic strand is fully complementary to the first region of the first nucleic acid strand, thereby forming no overhang at the 5′ and 3′ termini of the second nucleic acid strand in the first nucleic acid duplex.

In some embodiments, the second nucleic acid strand does not have an overhang at 3′ terminus, or 5′ terminus, or both in the first nucleic acid duplex. In some embodiments, the second nucleic acid strand comprises one or more phosphorothioate internucleoside linkages. In some embodiments, the second nucleic acid strand does not comprise phosphorothioate internucleoside linkages except for the internucleoside linkage(s) between the last two to three nucleosides at the 5′ terminus and the last two to three nucleosides at 3′ terminus. In some embodiments, the internucleoside linkage(s) between the last two, three or four nucleosides at the 5′ terminus of the second nucleic acid strand, the 3′ terminus of the second nucleic acid strand, or both, are phosphorothioate internucleoside linkages. In some embodiments, the 5′ terminus of the third nucleic acid strand comprises at least one phosphorothioate internucleoside linkage. In some embodiments, the last two, three or four nucleosides at the 5′ terminus of the third nucleic acid strand are phosphorothioate internucleoside linkages. In some embodiments, less than 5%, less than 10%, less than 25%, less than 50% of the internucleoside linkages in the first nucleic acid strand are phosphorothioate internucleoside linkages. In some embodiments, the first nucleic acid strand comprises no more than two phosphorothioate internucleoside linkages, or does not comprise phosphorothioate internucleoside linkages. In some embodiments, at least 50%, at least 60%, at least 70%, at least 80%, at least 85%, at least 90%, at least 95%, or all of the nucleosides of the first region of the first nucleic acid strand, the second region of the first nucleic strand, or both, are chemically modified.

At least 50%, at least 60%, at least 70%, at least 80%, at least 85%, at least 90%, at least 95%, or all of the nucleosides of one or more of the first nucleic acid strand, the second nucleic strand and the third nucleic strand can be chemically modified.

In some embodiments, at least 50%, at least 60%, at least 70%, at least 80%, at least 85%, at least 90%, at least 95%, or all of the nucleosides of the nucleic acid complex are chemically modified. In some embodiments, the chemical modifications are to resist nuclease degradation, to increase melting temperature (Tm), or both, of the nucleic acid complex. In some embodiments, at least 90%, at least 95%, or all of the nucleotides of the nucleic acid complex are non-DNA and non-RNA nucleotides. In some embodiments, at most 5%, at most 10%, or at most 15% of the nucleosides of the second nucleic strand are LNA. In some embodiments, about 10%-50% of the bases of the nucleic acid complex have a 2′-4′ bridging modifications. In some embodiments, about 10%-50% of the bases of the nucleic acid complex are locked nucleic acid (LNA) or analogues thereof. In some embodiments, about 10%-50% of the bases of the nucleic acid complex comprises 2′-O-methyl modification, 2′-F modification, or both. In some embodiments, the input nucleic acid strand is a RNA. In some embodiments, the target RNA is a cellular RNA transcript. In some embodiments, the target RNA is an mRNA, an miRNA, a non-coding RNA, a viral RNA transcript, or a combination thereof.

In some embodiments, the overhang of the second nucleic acid strand is capable of binding to the input nucleic acid strand to form a toehold, thereby causing the displacement of the second nucleic acid strand from the first nucleic acid strand. In some embodiments, the overhang of the third nucleic acid strand is 5 to 20 nucleosides in length. in some embodiments, the overhang of the third nucleic acid strand is 8 to 16 nucleosides in length, preferably 12 nucleosides in length. In some embodiments, the overhang of the third nucleic acid strand is 12 nucleotides in length. In some embodiments, all internucleoside linkages of the overhang of the third nucleic acid strand are phosphorothioate internucleoside linkages. In some embodiments, the 5′ terminus, the 3′ terminus, or both of the third nucleic acid strand comprises a terminal moiety. In some embodiments, the terminal moiety comprises a ligand, a fluorophore, a exonuclease, a fatty acid, a Cy3, an inverted dT attached to a tri-ethylene glycol, or a combination thereof.

Provided herein includes a method of modulating a target RNA, wherein the method includes: contacting a cell comprising a target RNA with any one or more of the nucleic acid complexes disclosed herein, wherein an input strand binds to the overhang of the third nucleic acid strand to cause displacement of the third nucleic acid strand from the first nucleic acid strand to release the sequence complementary to the target RNA into the cell, thereby modulating the target RNA. In some embodiments, contacting the cell with the nucleic acid complex is performed in vitro, in vivo, ex vivo, or a combination thereof. In some embodiments, contacting the cell with the nucleic acid complex occurs in the body of a subject. The cell can be, for example, a disease cell, and optionally the cell is a cancer cell. In some embodiments, the cell is a neuron.

Also provided includes a method of treating a disease or a condition, wherein the method includes administering one or more of the nucleic acid complexes disclosed herein to a subject in need thereof, wherein the input strand binds to the overhang of the third nucleic acid strand to cause displacement of the third nucleic acid strand from the first nucleic acid strand to release the sequence complementary to a target RNA, thereby reducing the activity of the target RNA or protein expression from the target RNA in the subject to treat the disease or condition. In some embodiments, the disease or condition is a central nervous system (CNS) disease or disorder or cancer. In some embodiments, the target RNA is a mRNA or a miRNA. In some embodiments, the nucleic acid complex is administered to a subject via a lipid-mediated delivery system, optionally via liposomes, nanoparticles, or micelles. In some embodiments, the nucleic acid complex is administered to a subject via nanoparticles, inorganic nanoparticles, nucleic acid lipid particles, polymeric nanoparticles, lipid nanoparticles (LNPs), chitosan and inulin nanoparticles, cyclodextrins nanoparticles, carbon nanotubes, liposomes, micellar structures, capsids, polymers, polymer matrices, hydrogels, dendrimers, nucleic acid nanostructure, exosomes, GalNAc-conjugated melittin-like peptides, or combinations thereof. In some embodiments, the nucleic acid complex is administered to a subject in need thereof via a subcutaneous injection. In some embodiments, the nucleic acid complex is administered to a subject in need thereof via an intravenous injection. In some embodiments, the nucleic acid complex is administered to a subject in need thereof at a concentration about 0.1-10 nM, optionally about 0.1-1 nM.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates a schematic representation of a non-limiting exemplary nucleic acid complex construct disclosed herein.

FIG. 2 illustrates a schematic representation of non-limiting exemplary nucleic acid complex constructs based on two designs.

FIG. 3 is a schematic diagram showing the formation of an active RNAi duplex following the displacement of a sensor nucleic acid strand from a core nucleic acid strand and the degradation of the core nucleic acid strand overhangs.

FIG. 4 shows sequence diagrams of three non-limiting exemplary nucleic acid complex constructs. Calc V3P3 passenger: SEQ ID NO: 7; Alt anp sens1: SEQ ID NO: 8; Alt anp-calc core 1: SEQ ID NO: 9; Alt anp sens2: SEQ ID NO: 10; Alt mus-calc core2: SEQ ID NO: 11; Alt mus-calc core 3: SEQ ID NO: 12.

FIG. 5A illustrates a schematic representation of a non-limiting exemplary nucleic acid complex construct TI CASi comprising a sensor strand, passenger strand and core strand. “+/−palmitic acid” indicates that palmitic acid can be optional at the terminus. FIGS. 5B-5D illustrate the chemical formulas of an exemplary sensor strand (FIG. 5B: SEQ ID NO: 6), passenger strand (FIG. 5C: SEQ ID NO: 1) and core strand (FIG. 5D: SEQ ID NO: 2), respectively. FIG. 5E shows the results from polyacrylamide gel electrophoresis (PAGE) analysis of TI CASi constructs, individual strands and duplexes. FIG. 5F shows an exemplary formulation of making a TI CASi construct.

FIG. 6 shows a graphic representation of various brain regions evaluated with the exemplary CASi constructs disclosed herein.

FIG. 7 depicts graphs showing the target mRNA levels in various brain regions of the mice treated with the TI CASi construct without a 3′ terminal palmitic acid (top panel) and with a 3′ terminal palmitic acid (bottom panel) 14 days after CASi injection.

FIG. 8 depicts a diagram showing the target mRNA levels in various brain regions of the mice treated with a CASi construct having a standard 8 nucleotide toehold (with or without palmitic acid) (“8 nt toe+PA” or “8 nt toe”), with a CASi construct having a 12 nucleotide toehold (12 nt toe), or with a CASi construct having a 16 nucleotide toehold (16 nt toe).

FIG. 9 depicts graphs showing the target mRNA levels in various brain regions of the mice treated with CASi constructs in comparison to saline treated mice 14 days, 30 days and 90 days after administration.

FIG. 10 depicts graphs showing the target mRNA levels in the spinal cord of the mice treated with the CASi constructs in comparison to saline treated mice 30 days and 90 days after the administration.

FIG. 11 depicts a graph showing the target mRNA levels in central and peripheral nervous systems 30 days after CASi administration.

FIG. 12 depict graphs showing the GFAP mRNA (top panel) and IBA-1 mRNA (bottom panel) levels in various brain regions of CASi treated animals (t) in comparison to saline treated animals (c).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

RNA interference (RNAi) is an intrinsic cellular mechanism conserved in most eukaryotes, that helps to regulate the expression of genes critical to cell fate determination, differentiation, survival and defense from viral infection. Researchers have exploited this natural mechanism by designing synthetic double-stranded RNA for sequence-specific gene silencing. Emerging developments in the field of dynamic nuclei acid nanotechnology and biomolecular computing also offer a conceptual approach to design programmable RNAi agents. However, challenges still remain in developing targeted RNAi therapy that can use nuclei acid logic switches to sense RNA transcripts (such as mRNAs and miRNAs) in order to restrict RNA silencing to specific populations of disease-related cells and spare normal tissues from toxic side effects. Significant challenges include poorly suppressed background drug activity, weak activated state drug potency, input and output sequence overlap, high design complexity, short lifetimes (<24 hours) and high required device concentrations (>10 nM).

Provided herein include conditionally activatable small interfering RNA (siRNA) complexes, components, compositions, and related methods and systems. The conditionally activatable siRNA complex can switch from an inactivated state to an activated state when triggered by a complementary binding of an input nucleic acid strand (e.g. a disease biomarker gene specific to disease-related cells) to the siRNA complex, thereby activating the RNA interference activity of the siRNA complex to target a specific target RNA (e.g. a RNA to be silenced). The nucleic acid complexes herein described can mediate conditionally activated RNA interference activity to silence target RNA in specific populations of disease-related cells with improved potency at a low concentration as well as improved specificity that can reduce off-target effects.

Disclosed herein includes a nucleic acid complex. The nucleic acid complex can comprise a first nucleic acid strand (e.g., core nucleic acid strand) comprising 20-60 linked nucleosides, a second nucleic acid strand (e.g., passenger nucleic acid strand) binding to a first region of the core nucleic acid strand to form a first nucleic acid duplex (e.g. RNAi duplex), and a third nucleic acid strand (e.g., sensor nucleic acid strand) binding to a second region of the core nucleic acid strand to form a second nucleic acid duplex (e.g. sensor duplex). The sensor nucleic acid strand can comprise an overhang that is not complementary to the core nucleic acid strand and is capable of binding to an input nucleic acid strand to cause the displacement of the third nucleic acid strand from the first nucleic acid strand. In some embodiments, the first region of the core nucleic acid strand is 3′ of the second region of the core nucleic acid strand. In some embodiments, the sensor nucleic acid strand does not bind to any region of the core nucleic acid strand that is 3′ of the first region of the core nucleic acid strand. In some embodiments, the first region of the core nucleic acid strand comprises a sequence complementary to a target RNA. The sequence complementary to a target RNA can be 10-35 nucleosides in length.

Disclosed herein also includes a method of modulating a target RNA. The method comprises contacting a cell comprising a target RNA with the nucleic acid complex herein described. Upon detection of an input nucleic acid strand, the input nucleic acid strand can bind to the overhang of the sensor nucleic acid strand to cause displacement of the sensor nucleic acid strand from the core nucleic acid strand to release the sequence complementary to the target RNA into the cell, thereby modulating the target RNA.

Disclosed herein also includes a method of treating a disease or a condition. The method comprises administering the nucleic acid complex herein described to a subject in need thereof. Upon detection of an input nucleic acid strand, the input strand can bind to the overhang of the sensor nucleic acid strand to cause displacement of the sensor nucleic acid strand from the core nucleic acid strand to release the sequence complementary to a target RNA, thereby reducing the activity of the target RNA or protein expression from the target RNA in the subject to treat the disease or condition.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide” and “nucleic acid molecule” are used interchangeably herein and refer to a polymer of nucleotides joined together by a phosphodiester linkage between 5′ and 3′ carbon atoms.

The terms “RNA”, “RNA molecule” and “ribonucleic acid molecule” refer to a polymer of ribonucleotides. The terms “DNA”, “DNA molecule” and deoxyribonucleic acid molecule” refer to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded or multi-stranded (e.g., double-stranded or triple-stranded). “mRNA” or “messenger RNA” is single-stranded RNA molecule that is complementary to one of the DNA strands of a gene. “miRNA” or “microRNA” is a small single-stranded non-coding RNA molecule that functions in RNA silencing and post-transcriptional regulation of gene expression.

The term “RNA analog” refers to an polynucleotide having at least one altered or modified nucleotide as compared to a corresponding unaltered or unmodified RNA. The nucleotide can retain the same or similar nature or function as the corresponding unaltered or unmodified RNA such as forming base pairs.

A single-stranded polynucleotide has a 5′ terminus or 5′ end and a 3′ terminus or 3′ end The terms “5′ end” “5′ terminus” and “3′ end” “3′ terminus” of a single-stranded polynucleotide indicate the terminal residues of the single-stranded polynucleotide and are distinguished based on the nature of the free group on each extremity. The 5′-terminus of a single-stranded polynucleotide designates the terminal residue of the single-stranded polynucleotide that has the fifth carbon in the sugar-ring of the deoxyribose or ribose at its terminus (5′ terminus). The 3′-terminus of a single-stranded polynucleotide designates the residue terminating at the hydroxyl group of the third carbon in the sugar-ring of the nucleotide or nucleoside at its terminus (3′ terminus). The 5′ terminus and 3′ terminus in various cases can be modified chemically or biologically e.g. by the addition of functional groups or other compounds as will be understood by the skilled person.

As used herein, the terms “complementary binding” and “bind complementarily” mean that two single strands are base paired to each other to form nucleic acid duplex or double-stranded nucleic acid. The term “base pair” as used herein indicates formation of hydrogen bonds between base pairs on opposite complementary polynucleotide strands or sequences following the Watson-Crick base pairing rule. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). A certain percentage of mismatches between the two single strands are allowed as long as a stable double-stranded duplex can be formed. In some embodiments, the two strands that bind complementarily can have a mismatches can be, about, be at most, or be at most bout 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

As used herein, the terms “RNA interference” “RNA interfering” and “RNAi” refer to a selective intracellular degradation of RNA. RNAi can occur in cells naturally to remove foreign RNAs (e.g., viral RNAs). Natural RNAi proceeds via fragments cleaved from free dsRNA which direct the degradative mechanism to other similar RNA sequences. RNAi can also be initiated recombinantly, for example, to silence the expression of target genes.

As used herein, the terms “small interfering RNA” and “siRNA” refer to an RNA or RNA analog capable of reducing or inhibiting expression of a gene or a target gene when the siRNA is activated in the same cell as the target gene. The siRNA used herein can comprise naturally occurring nucleic acid bases and/or chemically modified nucleic acid bases (RNA analogs).

### Nucleic Acid Complexes

Provided herein is a nucleic acid complex that can be conditionally activated upon a complementary binding to an input nucleic acid strand (e.g., a mRNA of a disease biomarker gene specific to a target cell, including a disease-related cell) through a sequence in a sensor nucleic acid strand of the nucleic acid complex. The activated nucleic acid complex can release the potent RNAi duplex formed by a core nucleic acid strand and a passenger nucleic acid strand, which can specifically inhibit or silence a target RNA. The target RNA can have a sequence independent from the input nucleic acid strand.

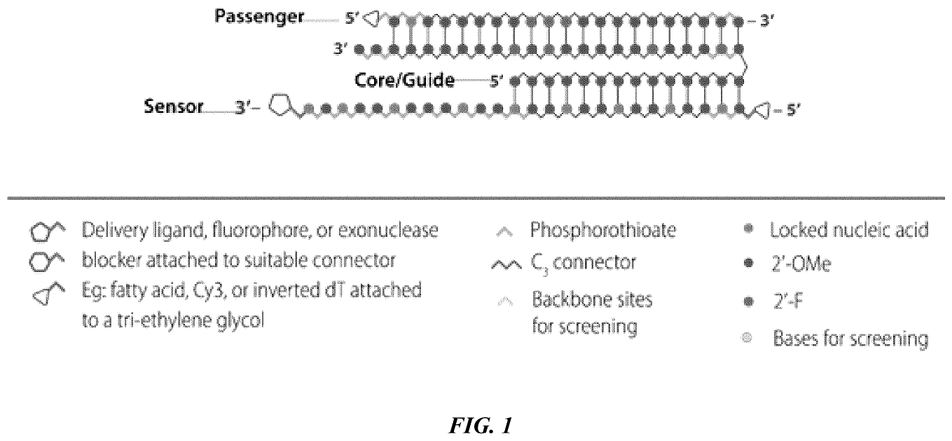

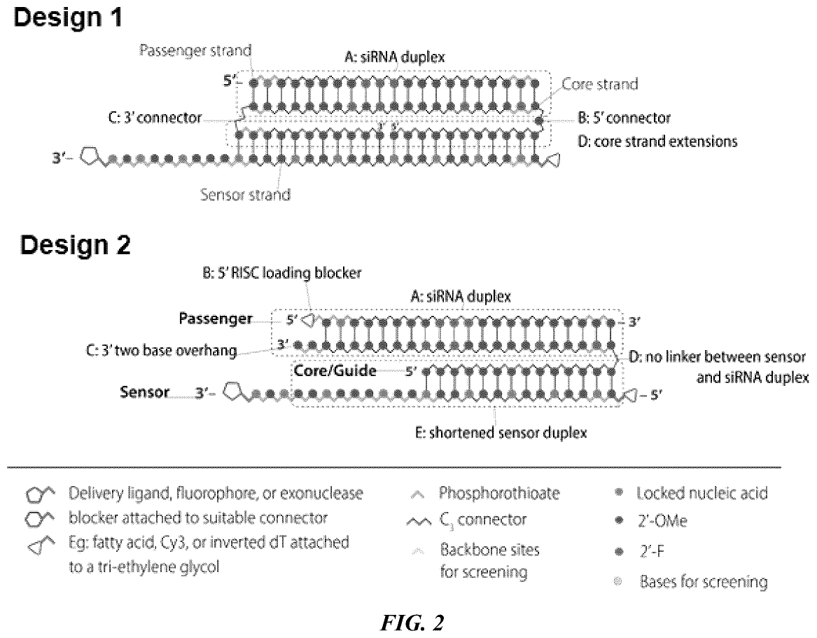

In some embodiments, the nucleic acid complexes described herein comprise a core nucleic acid strand (e.g. a first nucleic acid strand), a passenger nucleic acid strand (e.g. a second nucleic acid strand), and a sensor nucleic acid strand (e.g. a third nucleic acid strand) as shown in a non-limiting embodiment of FIG. 1. These three strands base-pair with one another to form a RNAi duplex (e.g. a first nucleic acid duplex) and a sensor duplex (e.g. a second nucleic acid duplex). The core nucleic acid strand, the passenger nucleic acid strand, and the sensor nucleic acid strand are RNA analogs comprising modified nucleotides.

The term “nucleic acid duplex” as used herein refers to two single-stranded polynucleotides bound to each other through complementarily binding. The nucleic acid duplex can form a helical structure, such as a double-stranded RNA molecule, which is maintained largely by non-covalent bonding of base pairs between the two single-stranded polynucleotides and by base stacking interactions.

The core nucleic acid strand can comprise a first region and a second region and the first region is at the 3′ direction of the second region. In other words, the first region is at the 3′ end of the core nucleic acid strand and the second region is at the 5′ end of the core nucleic acid strand. The first region of the core nucleic acid strand can be linked to the second region of the core nucleic acid strand via a connector, which can also be referred to as a 5′ connector. The 5′ connector can be a normal phosphodiester internucleoside linkage connecting two adjacent nucleotides. In some embodiments, the core nucleic acid strand only comprises one connector (e.g. 5′ connector) and does not comprise a 3′ connector.

The first region of the core nucleic acids strand is complementarily bound to the passenger nucleic acid strand to form a RNAi duplex (e.g. a first nucleic acid duplex). Not the entire sequence of the core nucleic acid strand is complementarily bound to the passenger nucleic acid strand. For example, the second region of the core nucleic acid strand is not complementarily bound to the passenger nucleic acid strand. In some embodiments, the first region of the core nucleic acid strand is fully complementary to the passenger nucleic acid strand, thereby forming a RNAi duplex having a blunt end with no overhang at the 5′ and 3′ termini of the first region of the core nucleic acid strand.

In some embodiments, the core nucleic acid strand of the RNAi duplex has a short overhang at the 3′ terminus (e.g. one, two, or three nucleosides), but the 3′ overhang does not extend back into the middle of the sensor duplex to bind with the sensor nucleic acid strand (see, for example, FIGS. 1-2). In some embodiments, the core nucleic acid strand does not have any region at the 3′ of the first region of the core nucleic acid strand.

The first region of the core nucleic acid strand can comprise a sequence complementary to a target nucleic acid (e.g. a RNA to be silenced). The core nucleic acid strand of the nucleic acid complex therefore acts as a guide strand (antisense strand) and is used to base pair with a target RNA. The passenger nucleic acid strand can therefore comprise a sequence homologous to the same target nucleic acid.

Upon activation of the nucleic acid complex (e.g. binding to an input nucleic acid strand), the released RNAi duplex can complementarily bind a target nucleic acid through the binding between the target nucleic acid and the first region of the core nucleic acid strand. In some embodiments, the sequence complementary to a target RNA in the core nucleic acid strand can be about 10-35 nucleosides in length. In some embodiments, the core nucleic acid strand comprises 20-60 linked nucleosides.

The sensor nucleic acid strand is complementarily bound to the second region of the core nucleic acid strand to form a sensor duplex (e.g. a second nucleic acid duplex). The sensor nucleic acid strand does not bind to the first region of the core nucleic acid strand nor any region of the core nucleic acid strand that is 3′ of the first region of the core nucleic acid strand. The sensor nucleic acid strand also does not bind to the passenger nucleic acid strand.

The sensor nucleic acid strand can comprise an overhang. The term “overhang” as used herein refers to a stretch of unpaired nucleotides that protrudes at one of the ends of a double-stranded polynucleotide (e.g. a duplex). An overhang can be on either strand of the polynucleotide and can be included at either the 3′ terminus of the strand (3′ overhang) or at the 5′ terminus of the strand (5′ overhang). The overhang can be at the 3′ terminus of the sensor nucleic acid strand. The overhang of the sensor nucleic acid strand does not bind to any region of the core nucleic acid strand. The overhang of the sensor nucleic acid strand can be about 5-20 nucleosides in length. In some embodiments, the overhang of the sensor nucleic acid strand is about 8-16 nucleosides in length, for example 8, 9, 10, 11, 12, 13, 14, 15, 16, or a number or a range between any two of these values, nucleotides in length. In some embodiments, the overhang of the sensor nucleic acid strand is 12 nucleosides in length.

The sensor nucleic acid strand can comprise a sequence capable of binding to an input nucleic acid strand (e.g. a disease biomarker gene specific to disease-related cells). Upon activation, the binding of the sensor nucleic acid strand to the input nucleic acid strand can cause displacement and subsequent release of the sensor nucleic acid strand from the core nucleic acid strand, thereby releasing the potent RNAi duplex and switching on the RNA interfering activity of the RNAi duplex. In the absence of an input nucleic acid strand or a detectable amount of the input nucleic acid strand, the nucleic acid complex herein described remains in an inactivated state (switched off) and the displacement of the sensor nucleic acid strand from the core nucleic acid strand does not take place. Therefore, the input nucleic acid strand can act as a trigger to activate (switch on) the RNA interfering activity of the nucleic acid complex (e.g. RNAi duplex).

The length of the RNAi duplex of the nucleic acid complex herein described can vary in different embodiments. In some embodiments, the length of the RNAi duplex can be 10-30 nucleotides. For example, the length of the RNAi duplex can be, 10, 11, 12, 13, 14, 15, 16, 17, 18, 19, 20, 21, 22, 23, 24, 25, 26, 27, 28, 29, or 30. In some embodiments, the length of the RNAi duplex can be 17-22 nucleotides.

The length of the sensor duplex of the nucleic acid complex herein described can vary in different embodiments. In some embodiments, the length of the sensor duplex can be 10-30 nucleotides. For example, the length of the sensor duplex can be, 10, 11, 12, 13, 14, 15, 16, 17, 18, 19, 20, 21, 22, 23, 24, 25, 26, 27, 28, 29, or 30. In some embodiments, the length of the sensor duplex is about 14 nucleotides. In some embodiments, the sensor duplex has a relatively short length with respect to the RNAi duplex.

In some embodiments, there is no linker molecule between a sensor duplex and a RNAi duplex of a nucleic acid complex except for the normal phosphodiester linkage connecting two adjacent nucleosides each located at a terminus of one of the two duplexes (see, for example, the CASi design shown in FIGS. 1-2).

An exemplary non-limiting embodiment of the nucleic acid complex herein described is illustrated in FIGS. 1-2. For example, the RNAi duplex formed by the passenger nucleic acid strand and the first region of the core nucleic acid strand has a length about 17-22 nucleotides (e.g. 21 nucleotides) and the sensor duplex formed by the second region of the core nucleic acid strand and the sensor nucleic acid strand has a length about 10-30 nucleotides (e.g. 14 nucleotides). The nucleotides of the nucleic acid complex can be extensively modified. For example, all of the nucleotides in the nucleic acid complex can be modified. The passenger nucleic acid strand can comprise two consecutive phosphorothioate internucleoside linkages at the 3′ terminus and three consecutive phosphorothioate internucleoside linkages at the 5′ terminus. The 5′ terminus of the passenger nucleic acid strand can be attached to a blocking moiety, such as a fluorophore, via a phosphorothioate internucleoside linkage to block interactions with RNAi pathway enzymes (e.g. Dicer, RISC). The 5′ terminus of the passenger nucleic acid strand can also comprise one or two LNA or analogues thereof. The core nucleic acid comprises a first region complementary bound to the passenger nucleic acid strand to form the RNAi duplex and a second region complementary bound to the sensor nucleic acid strand to form the sensor duplex. The first region can be longer than the second region, therefore rendering a shorter sensor duplex. The first region can be connected to the second region via a normal phosphodiester internucleoside linkage between two adjacent nucleotides with no additional connector or linker. The first region of the core nucleic acid strand can comprise two consecutive phosphorothioate internucleoside linkages at the 3′ terminus and two consecutive phosphorothioate internucleoside linkages at the 5′ terminus. The second region of the core nucleic acid strand can have no phosphorothioate internucleoside linkages. The core nucleic acid strand can comprise a 3′ overhang that is about two nucleotides in length. The overhang does not extend back into the middle of the sensor duplex to bind with the sensor nucleic acid strand. The sensor nucleic acid strand comprises a portion (e.g. 14 nucleotides) complementary bound to the second region of the core nucleic acid strand and a 3′ overhang (e.g. 12 nucleotides). All of the internucleoside linkages in the 3′ overhang of the sensor nucleic acid strand can be phosphorothioate internucleoside linkages. The 3′ terminus of the sensor nucleic acid strand can be attached to a terminal moiety such as a delivery ligand, a dye (e.g. fluorophore) or exonuclease. The 5′ terminus of the sensor nucleic acid strand can be attached to a terminal moiety such as a fatty acid, Cy3, or an inverted dT, a tri-ethylene glycol, or an inverted dT attached to a tri-ethylene glycol. The sensor nucleic acid strand can comprise a higher percentage of LNA, analogues thereof, or other 2′-4′ bridged bases than the passenger nucleic acid strand and the core nucleic acid strand. The core nucleic acid strand may comprise no LNA, analogues thereof, or other 2′-4′ bridged bases.

The nucleic acid complexes herein described can be synthesized using standard methods for oligonucleotide synthesis well-known in the art including, for example, Oligonucleotide Synthesis by Herdewijin, Piet (2005) and Modified oligonucleotides: Synthesis and Strategy for Users, by Verma and Eckstein, Annul Rev. Biochem. (1998): 67:99-134, the contents of which are incorporated herein by reference in their entirety. The synthesized nucleic acid complexes can be allowed to form its secondary structure under a desirable physiological condition as will be apparent to a skilled artisan. The formed secondary structure can be tested using standard methods known in the art such as chemical mapping, NMR, or computational simulations. The nucleic acid complex construct can be further modified, according to the test result, by introducing or removing chemical modifications or mismatches, as necessary, until the desired structure is obtained.

Suitable software suites can be used to aid in the design and analysis of nucleic acid structures. For example, Nupack can be used to check the formation of the duplexes and to rank the thermodynamic stability of the duplexes. Oligonucleotide design tools can be used to optimize the placement of LNA modifications.

### RNA Interference

Described herein are nucleic acid complexes that can be conditionally activated (e.g., via a signal for the presence of a mRNA of a gene specific for a target cell) to switch from an assembled, inactivated state to an activated state to act on (e.g. degrade or inhibit) a specific target nucleic acid in response to the detection of an input nucleic acid (e.g. nucleic acid specific to disease-related cells) having a sequence complementary to a sequence in the sensor nucleic acid strand of a nucleic acid complex.

In the assembled, inactivated configuration, the sensor nucleic acid strand of the nucleic acid complex inhibits enzymatic processing of the RNAi duplex, thereby keeping RNAi activity switched off.

In the event that an input nucleic acid strand complementary to the sensor nucleic acid strand of a nucleic acid complex is present, the input nucleic acid strand can activate the nucleic acid complex by inducing separation of the sensor nucleic acid strand from the core nucleic acid strand via toehold mediated strand displacement. Displacement can start from a toehold formed at the 3′ or 5′ terminus of the sensor nucleic acid strand (e.g. a toehold formed at the 3′ terminus of the sensor nucleic acid strand) through a complementary binding between the input nucleic acid strand and an overhang of the sensor nucleic acid strand.

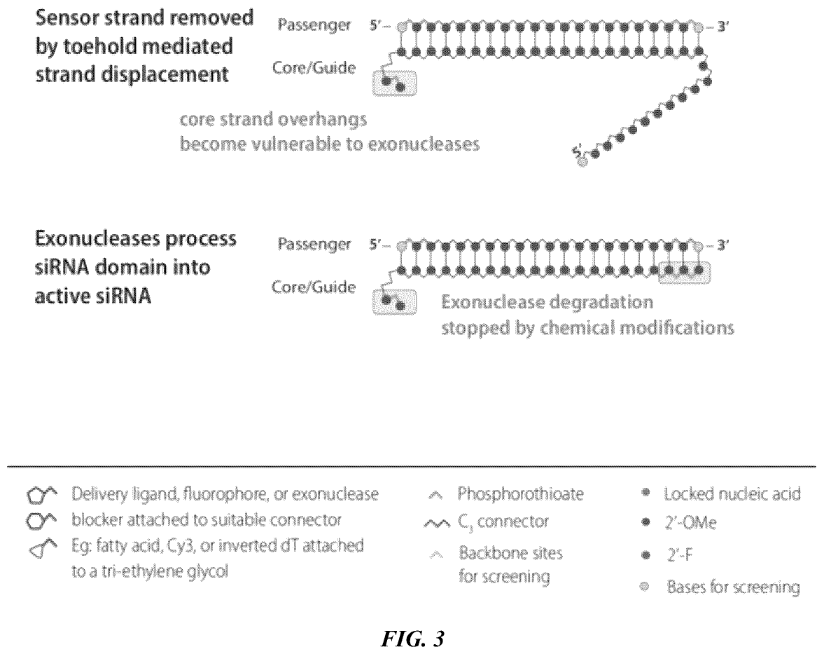

After removal of the sensor nucleic acid strand, the second region of the core nucleic acid strand becomes a 5′ overhang that can be degraded by nucleases (e.g. exonuclease). This degradation stops at the 5′ end of the RNAi duplex due to the presence of chemically modified nucleotides and/or exonuclease cleavage-resistance moieties, thus rendering an active RNAi duplex for further endonuclease processing if needed and RNA-induced silencing complex (RISC) loading. RISC is a multiprotein complex that incorporates one strand of a siRNA or miRNA and uses the siRNA or miRNA as a template for recognizing complementary target nucleic acid. Once a target nucleic acid is identified, RISC activates RNase (e.g. Argonaute) and inhibits the target nucleic acid by cleavage. In some embodiments, Dicer is not required for loading the RNAi duplex into RISC. FIG. 3 is a schematic diagram showing the formation of an active RNAi duplex following the displacement of a sensor nucleic acid strand from a core nucleic acid strand and the degradation of the core nucleic acid strand overhangs.

The passenger nucleic acid strand is then discarded, while the core nucleic acid strand (the first region of the core nucleic acid strand) is incorporated into RICS. The core nucleic acid strand of the nucleic acid complex disclosed herein acts as a guide strand (antisense strand) and is used to base pair with a target RNA. The passenger nucleic acid strand acts as a protecting strand prior to the loading of the core nucleic acid strand into RICS. RICS uses the incorporated core nucleic acid strand as a template for recognizing a target RNA that has complementary sequence to the core nucleic acid strand, particularly the first region of the core nucleic acid strand. Upon binding to the target RNA, the catalytic component of RICS, Argonaute, is activated which can degrade the bound target RNA. The target RNA can be degraded or the translation of the target RNA can be inhibited.

In some embodiments, the nucleic acid complexes herein described do not have a dicer cleavage site, and therefore the RNAi interference mediated by the nucleic acid complexes can bypass Dicer-mediated cleavage.

As will be apparent to a skilled artisan, Dicer is an endoribonuclease in the RNAse III family that can initiate the RNAi pathway by cleaving double-stranded RNA (dsRNA) molecule into short fragments of dsRNAs about 20-25 nucleotides in length.

In some embodiments, the nucleic acid complexes herein described differentiate from the conditionally activated small interfering RNAs (Cond-siRNAs) disclosed in the related international application published as WO/2020/033938 in that the nucleic acid complexes herein described can bypass the Dicer processing.

In some embodiments, the nucleic acid complexes disclosed herein have structural features that discourage the Dicer binding. In some embodiments, the RNAi duplex does not create a Dicer substrate. For example, the RNAi duplex formed by the passenger nucleic acid strand and the first region of the core nucleic acid strand do not have a 3′ and/or 5′ overhang, but instead forming a blunt end that can render the passenger nucleic acid strand unfavorable for Dicer binding. In some embodiments, the passenger nucleic acid strand has about 17-22 nucleotides in length, making it short enough to bypass Dicer cleavage. In some embodiments, the passenger nucleic acid strand does not have G/C rich bases to the 3′ and/or 5′ end of the passenger nucleic acid strand. In some embodiments, the passenger nucleic acid strand is attached to a terminal moiety and/or a blocking moiety to avoid Dicer binding.

Upon activation, the nucleic acid complex herein described can inhibit a target nucleic acid in target cells, therefore resulting in a reduction or loss of expression of the target nucleic acid in the target cells. The target cells are cells associated or related to a disease or disorder. The term “associated to” “related to” as used herein refers to a relation between the cells and the disease or condition such that the occurrence of a disease or condition is accompanied by the occurrence of the target cells, which includes but is not limited to a cause-effect relation and sign/symptoms-disease relation. The target cells used herein typically have a detectable expression of an input nucleic acid.

In some embodiments, the expression of a target nucleic acid in target cells is inhibited by about, at least, or at least about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any of these values.

As used herein, inhibition of gene expression refers to the absence or observable decrease in the level of protein and/or mRNA product from a target gene in target cells. The degree of inhibition can be evaluated by examination of the expression level of the target gene as demonstrated in the examples.

In some embodiments, gene expression can be assayed by use of a reporter or drug resistance gene whose protein product is easily assayed. Exemplary reporter genes include, but no limiting to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarmicin, hygromycin, kanamycin, lincomycin, methotrexate, phosphinothricin, puromycin, and tetracycline. Quantitation of the amount of gene expression allows one to determine a degree of inhibition as compared to cells not treated with the nucleic acid complexes or treated with a negative or positive control. Various biochemical techniques may be employed as will be apparent to a skilled artisan such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

In some embodiments, the nucleic acid complexes disclosed herein exhibit improved switching performance and reduced off-target effects. The nucleic acid complexes disclosed herein can have a reduced unwanted RNAi activity when the nucleic acid complexes are in an inactivated state (switched off) and an enhanced RNAi activity when the nucleic acid complexes are activated upon detection of an input nucleic acid strand.

In some embodiments, the expression of a target nucleic acid in non-target cells (e.g. cells not having an input nucleic acid strand) is inhibited about, at most, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any of these values. Non-target cells can comprise cells of the subject other than target cells.

In some embodiments, the nucleic acid complexes herein described have an enhanced potency, thus capable of evoking an RNAi activity at low concentrations. Nonspecific, off-target effects and toxicity (e.g. undesired proinflammatory responses) can be minimized by using low concentrations of the nucleic acid complexes.

The concentration of the nucleic acid complexes disclosed herein can vary in different embodiments. In some embodiments, the nucleic acid complexes disclosed herein can be provided at a concentration of, about, at most, or at most about, 0.001 nM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, 11 nM, 12 nM, 13 nM, 14 nM, 15 nM, 16 nM, 17 nM, 18 nM, 19 nM, 20 nM, 30 nM, 40 nM, 50 nM, or a number or a range between any two of these values. For example, the nucleic acid complexes disclosed herein can be provided at a concentration between about 0.1-10 nM, preferably between about 0.1-1 nM. In some embodiments, the nucleic acid complex herein disclosed has a transfection concentration at 0.1 nM or lower.

The nucleic acid complex herein described can allow lasting and consistently potent inhibition effects at low concentrations. For example, the nucleic acid complex can remain active for an extended period of time such as 1 hour, 2 hours, 6 hours, 12 hours, 24 hours, 36 hours, 48 hours, 60 hours, 72 hours, 84 hours, 96 hours, 5 days, 6 days, 7 days, two weeks, or a number or a range between any of these values, or more. In some embodiments, the nucleic acid complex can remain active for at least 12 hours, at least 24 hours, at least 36 hours, at least 48 hours, at least 60 hours, at least 72 hours, at least 84 hours, or at least 96 hours. In some embodiments, the nucleic acid complex can remain active for up to 30 days, up to 60 days, or up to 90 days.

### Chemical Modification

The nucleic acid strands (the core nucleic acid strand, the passenger nucleic acid strand, and/or the sensor nucleic acid strand) comprised in the nucleic acid complexes herein described can be a non-standard, modified nucleic acid strand comprising non-standard, modified nucleotides (nucleotide analog) or non-standard, modified nucleosides (nucleoside analog). The term “nucleotide analog” or “modified nucleotide” refers to a non-standard nucleotide comprising one or more modifications (e.g. chemical modifications), including non-naturally occurring ribonucleotides or deoxyribonucleotides. The term “nucleoside analog” or “modified nucleoside” refers to a non-standard nucleoside comprising one or more modification (e.g. chemical modification), including non-naturally occurring nucleosides other than cytidine, uridine, adenosine, guanosine, and thymidine. The modified nucleoside can be a modified nucleotide without a phosphate group. The chemical modifications can include replacement of one or more atoms or moieties with a different atom or a different moiety or functional group (e.g. methyl group or hydroxyl group).

The modifications are introduced to alter certain chemical properties of the nucleotide/nucleoside such as to increase thermodynamic stability, to increase resistance to nuclease degradation (e.g. exonuclease resistant), and/or to increase binding specificity and minimize off-target effects. For example, thermodynamic stability can be determined based on measurement of melting temperature Tₘ. A higher Tₘ can be associated with a more thermodynamically stable chemical entity.

In some embodiments, the modification can render one or more of the nucleic acid strands in the nucleic acid complex to resist exonuclease degradation/cleavage. The term “exonuclease” as used herein, indicates a type of enzyme that works by cleaving nucleotides one at a time from the end (exo) of a polynucleotide chain. A hydrolyzing reaction that breaks phosphodiester bonds at either the 3′ or the 5′ end occurs. A 3′ and 5′ exonuclease can degrade RNA and DNA in cells, and can degrade RNA and DNA in the interstitial space between cells and in plasma, with a high efficiency and a fast kinetic rate. A close relative is the endonuclease, which cleaves phosphodiester bonds in the middle (endo) of a polynucleotide chain. 3′ and 5′ exonuclease and exonucleolytic complexes can degrade RNA and DNA in cells, and can degrade RNA and DNA in the interstitial space between cells and in plasma. The term “exoribonuclease” as used herein, refers to exonuclease ribonucleases, which are enzymes that degrade RNA by removing terminal nucleotides from either the 5′ end or the 3′ end of the RNA molecule. Enzymes that remove nucleotides from the 5′ end are called 5′-3′ exoribonucleases, and enzymes that remove nucleotides from the 3′ end are called 3′-5′ exoribonucleases.

The modification can comprise phosphonate modification, ribose modification (in the sugar portion), and/or base modification. Preferred modified nucleotides used herein include sugar- and/or backbone-modified ribonucleotides.

In some embodiments, the modified nucleotide can comprise modifications to the sugar portion of the nucleotides. For example, the 2′ OH-group of a nucleotide can be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. In some embodiments, the 2′ OH-group of a nucleotide or nucleoside is replaced by 2′ O-methyl group and the modified nucleotide or nucleoside is a 2′-O-methyl nucleotide or 2′-O-methyl nucleoside (2′-OMe). The 2′-O-methyl nucleotide or 2′-O-methyl nucleoside can be 2′-O-methyladenosine, 2′-O-methylguanosine, 2′-O-methyluridine, or 2′-O-methylcytidine. In some embodiments, the 2′ OH-group of a nucleotide is replaced by fluorine (F), and the modified nucleotide or nucleoside is a 2′-F nucleotide or 2′-F nucleoside (2′-deoxy-2′-fluoro or 2′-F). The 2′-F nucleotide or 2′-F nucleoside can be 2′-F-adenosine, 2′-F-guanosine, 2′-F-uridine, or 2′-F-cytidine. The modifications can also include other modifications such as nucleoside analog phosphoramidites. In some embodiments, glycol nucleic acids can be used.

In some embodiments, the modified nucleotide can comprise a modification in the phosphate group of the nucleotide, e.g. by substituting one or more of the oxygens of the phosphate group with sulfur or a methyl group. In some embodiments, one or more of the nonbridging oxygens of the phosphate group of a nucleotide is replaced by a sulfur.

In some embodiments, the nucleic acid strands herein described comprise one or more non-standard internucleoside linkage that is not a phosphodiester linkage. In some embodiments, the nucleic acid strands herein described comprise one or more phosphorothioate internucleoside linkages. The term “phosphorothioate linkage” (PS) as used herein, indicates a bond between nucleotides in which one of the nonbridging oxygens is replaced by a sulfur. In some embodiments, both nonbridging oxygens may be replaced by a sulfur (PS2). In some embodiments, one of the nonbridging oxygens may be replaced by a methyl group. The term “phosphodiester linkage” as described herein indicates the normal sugar phosphate backbone linkage in DNA and RNA wherein a phosphate bridges the two sugars. In some embodiments, the introduction of one or more phosphorothioate linkage in the core nucleic acid strand, the passenger nucleic acid strand, and/or the sensor nucleic acid strand can endow the modified nucleotides with increased resistance to nucleases (e.g. endonucleases and/or exonucleases).

In some embodiments, the modified nucleotide can comprise modifications to or substitution of the base portion of a nucleotide. For example, uridine and cytidine residues can be substituted with pseudouridine, 2-thiouridine, N6-methyladenosine, 5-methycytidine or other base analogs of uridine and cytidine residues. Adenosine can comprise modifications to Hoogsteen (e.g., 7-triazolo-8-aza-7-deazaadenosines) and/or Watson-Crick face of adenosine (e.g. N2-alkyl-2-aminopurines). Examples of adenosine analogs also include Hoogsteen or Watson-Crick face-localized N-ethylpiperidine triazole-modified adenosine analogs, N-ethylpiperidine 7-EAA triazole (e.g., 7-EAA, 7-ethynyl-8-aza-7-deazaadenosine) and other adenosine analogs identifiable to a person skilled in the art. Cytosine may be substituted with any suitable cytosine analogs identifiable to a person skilled in the art. For example, cytosine can be substituted with 6′-phenylpyrrolocytosine (PhpC) which has shown comparable base pairing fidelity, thermal stability and high fluorescence.

In some embodiments, one or more nucleotides in the nucleic acid complex disclosed herein can be substituted with a universal base. The term “universal base” refers to nucleotide analogs that form base pairs with each of the natural nucleotides with little discrimination between them. Examples of universal bases include, but are not limited to, C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see e.g., Loakes, 2001, Nucleic Acids Research, 29, 2437-2447).

In some embodiments, base modification disclosed herein can reduce innate immune recognition while making the nucleic acid complex more resistant to nucleases. Examples of base modifications that can be used in the nucleic acid complex disclosed herein are also described, for example, in Hu et al. (Signal Transduction and targeted Therapy 5:101 (2020)), the content of which is incorporated by reference in its entirety.

In some embodiments, the nucleic acid strands (the core nucleic acid strand, the passenger nucleic acid strand, and/or the sensor nucleic acid strand) comprised in the nucleic acid complexes herein described can comprise one or more locked nucleic acids or analogs thereof. Exemplary locked nucleic acid analogs include, for example, their corresponding locked analog phosphoramidites and other derivatives apparent to a skilled artisan.

As used herein, the term “locked nucleic acids” (LNA) indicates a modified RNA nucleotide. The ribose moiety of an LNA nucleotide is modified with an extra bridge connecting the 2′ and 4′ carbons (a 2′-0, 4′-C methylene bridge). The bridge “locks” the ribose in the 3′-endo structural conformation and restricts the flexibility of the ribofuranose ring, thereby locking the structure into a rigid bicyclic formation. LNA nucleotides can be mixed with DNA or RNA bases in the oligonucleotide whenever desired. The incorporation of LNA into the nucleic acid complexes disclosed herein can increase the thermal stability (e.g. melting temperature), hybridization specificity of oligonucleotides as well as accuracies in allelic discrimination. LNA oligonucleotides display hybridization affinity toward complementary single-stranded RNA and complementary single- or double-stranded DNA. Additional information about LNA can be found, for example, at www.sigmaaldrich.com/technical-documents/articles/biology/locked-nucleic-acids-faq.html. In some embodiments, glycol nucleic acids can be used.

In some embodiments, the nucleic acid strands (the core nucleic acid strand, the passenger nucleic acid strand, and/or the sensor nucleic acid strand) comprised in the nucleic acid complexes herein described can comprise other chemically modified nucleotide or nucleoside with 2′-4′ bridging modifications (e.g. 2′-4′ bridged bases). A 2′-4′ bridging modification refers to the introduction of a bridge connecting the 2′ and 4′ carbons of a nucleotide. The bridge can be a 2′-0, 4′-C methylene bridge (e.g. in LNA). The bridge can also be a 2′-O, 4′-C ethylene bridge (e.g. in ethylen-bridged nucleic acids (ENA)) or any other chemical linkage identifiable to a person skilled in the art.

In some embodiments, the introduction of LNA, analogues thereof, or other chemically modified nucleotides with 2′-4′ bridging modifications in the nucleic acid complex herein described can enhance hybridization stability as well as mismatch discrimination. For example, a nucleic acid complex comprising a sensor nucleic acid strand with LNA, analogues thereof, or other chemically modified nucleotides with 2′-4′ bridging modifications can have an enhanced sensitivity to distinguish between matched and mismatched input nucleic acid strand (e.g. in the complementary binding between an input nucleic acid strand and a sensor nucleic acid strand).

In some embodiments, one or more of the nucleic acid strands of the nucleic acid complex can comprise a chemical moiety linked to the 3′ and/or 5′ terminus of the strand. The terminal moiety can include one or more suitable terminal linkers or modifications. For example, the terminal moiety can include a linker to link the oligonucleotide with another molecule or a particular surface (biotins, amino-modifiers, alkynes, thiol modifiers, azide, N-Hydroxysuccinimide, and cholesterol), a dye (e.g. fluorophore, Cy3, or a dark quencher), a fluorine modified ribose, a space (e.g. C3 spacer, Spacer 9, Spacer 18, dSpacer, tri-ethylene glycol spacer, hexa-ethylene glycol spacer), moieties and chemical modification involved in click chemistry (e.g. alkyne and azide moieties), and any linkers or terminal modifications that can be used to attach the 3′ and 5′ end to other chemical moieties such as antibodies, gold or other metallic nanoparticles, polymeric nanoparticles, dendrimer nanoparticles, small molecules, single chain or branched fatty acids, peptides, proteins, aptamers, and other nucleic acid strands and nucleic acid nanostructures. The terminal moiety can serve as a fluorescent label used for imaging and detection. The terminal moiety can also comprise one or more exonuclease cleavage-resistant group or moiety to protect a single-stranded nucleic acid from nuclease degradation (e.g. exonuclease cleavage-resistant moiety). In some embodiments, the terminal moiety can also serve as a blocking moiety to block interactions with RNAi pathway enzymes such as Dicer and RISC. The terminal moiety can also comprise a ligand suitable for nucleic acid delivery and for use in targeting the nucleic acid complex to specific cell types described elsewhere in the present disclosure. Additional linkers and terminal modification that can be attached to the terminus of the sensor nucleic acid strand are described in www.idtdna.com/pages/products/custom-dna-rna/oligo-modifications and www.glenresearch.com/browse/labels-and-modifiers, the contents of which are incorporated herein by reference in their entirety. Examples of exonuclease blocking moieties that can be used in the composition disclosed herein are described, for example, in international patent application published as WO/2020/033938, the content of which is incorporated herein by reference in its entirety.

Additional modifications to the nucleotides and/or nucleosides can also be introduced to one or more strands of the nucleic acid complex herein described, such as modifications described in Hu et al. (Signal Transduction and targeted Therapy 5: 101 (2020)), the content of which is incorporated by reference in its entirety.

### Ribose Modification

The percentage of the modified nucleosides of the nucleic acid complex can vary in different embodiments. In some embodiments, the percentage of the modified nucleosides of the nucleic acid complex herein described can be, be about, be at least, or be at least about 50%, 60%, 70%, 80%, 85%, 90%, or 95%. For example, percentage of the modified nucleosides of the nucleic acid complex herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, at least 90%, 91%, 92%, 93%, 94%, 95%, or a number or a range between any two of these values of the nucleotides of the nucleic acid complex are modified (e.g. are non-DNA and non-RNA). In some embodiments, all of the nucleotides of the nucleic acid complex are modified (e.g. are non-DNA and non-RNA).

The percentage of the modified nucleosides in one or more strands of the nucleic acid complex can vary in different embodiments. In some embodiments, the percentage of the modified nucleosides in a core nuclei acid strand herein described can be, be about, be at least, or be at least about 50%, 60%, 70%, 80%, 85%, 90%, or 95%. For example, the percentage of the modified nucleosides in a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a core nucleic acid strand are chemically modified.

In some embodiments, the percentage of the modified nucleosides in the first region of a core nuclei acid strand herein described can be, be about, be at least, or be at least about 50%, 60%, 70%, 80%, 85%, 90%, or 95%. For example, the percentage of the modified nucleosides in the first region of a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the first region of a core nucleic acid strand are chemically modified.

The percentage of the modified nucleosides in the second region of a core nuclei acid strand herein described can be, be about, be at least, or be at least about 50%, 60%, 70%, 80%, 85%, 90%, or 95%. For example, the percentage of the modified nucleosides in the second region of a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the second region of a core nucleic acid strand are chemically modified.

The percentage of the modified nucleosides in a passenger nuclei acid strand herein described can be, be about, be at least, or be at least about 50%, 60%, 70%, 80%, 85%, 90%, or 95%. For example, the percentage of the modified nucleosides in a passenger nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a passenger nucleic acid strand are chemically modified.

In some embodiments, the percentage of the modified nucleosides in a sensor nuclei acid strand herein described can be, be about, be at least, or be at least about 50%, 60%, 70%, 80%, 85%, 90%, or 95%. For example, the percentage of the modified nucleosides in a sensor nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a sensor nucleic acid strand are chemically modified.

The modified nucleosides in one or more of the core nucleic acid strand, the passenger nucleic acid strand, and the sensor nucleic acid strand can comprise 2′-O-methyl nucleoside and/or 2′-F nucleoside.

In some embodiments, the percentage of 2′-O-methyl nucleoside and/or 2′-F nucleoside in the nucleic acid complex herein described can be, be about, be at least, be at least about, be at most, or be at most about 10%-50%. For example, the percentage of 2′-O-methyl nucleoside and/or 2′-F nucleoside in the nucleic acid complex herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

The percentage of 2′-O-methyl nucleoside and/or 2′-F nucleoside in a core nucleic acid strand herein described can be, be about, be at least, be at least about, be at most, or be at most about 10%-50%. For example, the percentage of 2′-O-methyl nucleoside and/or 2′-F nucleoside in a core nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a passenger nucleic acid strand herein described can be, be about, be at least, be at least about, be at most, or be at most about 10%-50%. For example, the percentage of 2′-O-methyl nucleoside and/or 2′-F nucleoside in a passenger nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a sensor nucleic acid strand herein described can be, be about, be at least, be at least about, be at most, or be at most about 10%-50%. For example, the percentage of 2′-O-methyl nucleoside and/or 2′-F nucleoside in a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

### Phosphate Modification

The percentage of phosphate modification to the nucleotides in the nucleic acid complex described herein can vary in different embodiments. In some embodiments, the phosphate modification comprises or is a phosphorothioate internucleoside linkage. In some embodiments, the percentage of phosphorothioate internucleoside linkages in a core nucleic acid strand is less than 5%, less than 10%, less than 25%, less than 50%, or a number or a range between any two of these values. For example, percentage of phosphorothioate internucleoside linkages in a core nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values. In some embodiments, the core nucleic acid strand comprises no more than two phosphorothioate internucleoside linkages. In some embodiments, the core nucleic acid strand does not comprise a phosphorothioate internucleoside linkage modification.

The percentage of phosphodiester internucleoside linkages in a core nucleic acid strand can be about, at least, or at least about 50%, 80% or 95%, or a number or a range between any two of these values. For example, percentage of phosphodiester internucleoside linkages in a core nucleic acid strand is about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, all the internucleoside linkages in the core nucleic acid strand are phosphodiester internucleoside linkage.

In some embodiments, the 3′ terminus of the first region of the core nucleic acid strand comprises at least one phosphorothioate internucleoside linkage (e.g. one, two or three phosphorothioate internucleoside linkage). The phosphorothioate internucleoside linkage can be between the last two, three, or four nucleosides at the 3′ terminus of the first region of the core nucleic acid strand. In some embodiments, the 5′ terminus of the first region of the core nucleic acid strand comprises at least one phosphorothioate internucleoside linkage (e.g. one, two or three phosphorothioate internucleoside linkage). The phosphorothioate internucleoside linkage can be between the last two, three, or four nucleosides at the 5′ terminus of the first region of the core nucleic acid strand. In some embodiments, each of the 5′ terminus of the first region of the core nucleic acid strand and the 3′ terminus of the first region of the core nucleic acid strand independently comprises one or more phosphorothioate internucleoside linkages (e.g. one, two or three phosphorothioate internucleoside linkage). In some embodiments, the first region of the core nucleic acid strand does not comprise phosphorothioate internucleoside linkages except for the phosphorothioate internucleoside linkage(s) between the last two or three nucleosides at the 5′ terminus, 3′ terminus, or both, of the first region. For example, the first region of the core nucleic acid strand does not comprise phosphorothioate internucleoside linkages except for the phosphorothioate internucleoside linkage(s) between the last three nucleosides at the 5′ terminus and the last three nucleosides 3′ terminus of the first region.

In some embodiments, the percentage of phosphorothioate internucleoside linkages in the second region of a core nucleic acid strand is less than 5%, less than 10%, or a number or a range between any two of these values. In some embodiments, the second region of a core nucleic acid strand does not comprise phosphorothioate internucleoside linkages.

In some embodiments, the passenger nucleic acid strand comprises one or phosphorothioate internucleoside linkage. The percentage of phosphorothioate more internucleoside linkages in a passenger nucleic acid strand can be less than 5%, less than 10%, less than 25%, less than 50%, or a number or a range between any two of these values. For example, percentage of phosphorothioate internucleoside linkages in a passenger nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

In some embodiments, the 5′ terminus of the passenger nucleic acid strand comprises one or more phosphorothioate internucleoside linkage (e.g. one, two, or three phosphorothioate internucleoside linkage). In some embodiments, the 3′ terminus of the passenger nucleic acid strand comprises at least one phosphorothioate internucleoside linkage (e.g. one, two, or three phosphorothioate internucleoside linkage). In some embodiments, each of the 5′ terminus of the passenger nucleic acid strand and the 3′ terminus of the passenger nucleic acid strand independently comprises one or more phosphorothioate internucleoside linkages (e.g. one, two or three phosphorothioate internucleoside linkage). In some embodiments, the passenger nucleic acid strand does not comprise phosphorothioate internucleoside linkages except for the phosphorothioate internucleoside linkage(s) between the last two, three, or four nucleosides at the 5′ terminus, 3′ terminus, or both, of the passenger nucleic acid strand. In some embodiments, the passenger nucleic acid strand does not comprise phosphorothioate internucleoside linkages except for the phosphorothioate internucleoside linkage(s) between the last two to three nucleosides at the 5′ terminus and the last two to three nucleosides at 3′ terminus of the passenger nucleic acid strand.

The sensor nucleic acid strand can comprise one or more phosphorothioate internucleoside linkage. The percentage of phosphorothioate internucleoside linkages in a sensor nucleic acid strand can be less than 5%, less than 10%, less than 25%, less than 50%, less than 60%, less than 70% or a number or a range between any two of these values. For example, percentage of phosphorothioate internucleoside linkages in a sensor nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or a number or a range between any two of these values.

In some embodiments, the 5′ terminus of the sensor nucleic acid strand comprises at least one phosphorothioate internucleoside linkage (e.g. one, two or three phosphorothioate internucleoside linkage). In some embodiments, the 3′ terminus of the sensor nucleic acid strand comprises at least one phosphorothioate internucleoside linkage (e.g. one to twenty phosphorothioate internucleoside linkage. In some embodiments, each of the 5′ terminus of the sensor nucleic acid strand and the 3′ terminus of the sensor nucleic acid strand independently comprises one or more phosphorothioate internucleoside linkages (e.g. one, two or three at the 5′ terminus or one to twenty at the 3′ terminus). In some embodiments, the sensor nucleic acid strand does not comprise phosphorothioate internucleoside linkages except for the phosphorothioate internucleoside linkage(s) at the 5′ terminus, 3′ terminus, or both, of the passenger nucleic acid strand. In some embodiments, the phosphorothioate internucleoside linkages at the 3′ terminus of the passenger nucleic acid strand are in the singled-stranded overhang of the passenger nucleic acid strand.

### LNA, Analogues Thereof and 2′-4′Bridging Modification

The percentage of the LNA or analogues thereof of the nucleic acid complex can vary in different embodiments. In some embodiments, the percentage of the LNA or analogues thereof of the nucleic acid complex herein described can be about 10%-50%. For example, the percentage of the LNA or analogues thereof of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

The percentage of the LNA or analogues thereof in one or more strands of the nucleic acid complex can vary in different embodiments. In some embodiments, the percentage of the LNA or analogues thereof in a core nucleic acid strand herein described can be, be about, be at most, or be at most about 5%, 10%, or 15%. For example, the percentage of the LNA or analogues thereof of a core nucleic acid strand herein described can be, be about, be at most, or be at most about 5%, 6%, 7%, 8%, 9%, 10%, 11%, 12%, 13%, 14%, 15%, or a number or a range between any two of these values.

In some embodiments, the percentage of the LNA or analogues thereof in a passenger nucleic acid strand herein described can be, be about, be at most, or be at most about 5%, 10%, or 15%. For example, the percentage of the LNA or analogues thereof of a passenger nucleic acid strand herein described can be, be about, be at most, or be at most about 5%, 6%, 7%, 8%, 9%, 10%, 11%, 12%, 13%, 14%, 15%, or a number or a range between any two of these values. In some embodiments, a percentage of the LNA or analogues thereof in a passenger nucleic acid strand herein described greater than 5%, greater than 10%, or greater than 15% can decrease the RNAi activity of the nucleic acid complex.

In some embodiments, the percentage of the LNA or analogues thereof in a sensor nucleic acid strand herein described can be, be about, be at least, be at least about, be at most, or be at most about 10%-50%. For example, the percentage of the LNA or analogues thereof of a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

The percentage of 2′-4′ bridging modification of the nucleic acid complex can vary in different embodiments. In some embodiments, the percentage of the 2′-4′ bridging modification of the nucleic acid complex herein described can be about 10%-50%. For example, the percentage of the 2′-4′ bridging modification of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

### Core Strand

The core nucleic acid strand of the nucleic acid complex described herein can comprise a first region and a second region. The first region is at the 3′ direction of the second region.

The length of the core nucleic acid strand can vary. In some embodiments, the core nucleic acid strand comprises 20-60 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, linked nucleosides. The length of the first region of the core nucleic acid strand can vary. In some embodiments, the first region of the core nucleic acid strand comprises 10-30 linked nucleosides. For example, the first region of the core nucleic acid strand can comprise 10, 11, 12, 13, 14, 15, 16, 17, 18, 19, 20, 21, 22, 23, 24, 25, 26, 27, 28, 29, or 30, linked nucleosides. In some embodiments, the first region of the core nucleic acid strand comprises 17-22 linked nucleosides.

The length of the second region of the core nucleic acid strand can vary in different embodiments. In some embodiments, the length of the second region of the core nucleic acid strand comprises 10-30 linked nucleosides. For example, the second region of the core nucleic acid strand can comprise 10, 11, 12, 13, 14, 15, 16, 17, 18, 19, 20, 21, 22, 23, 24, 25, 26, 27, 28, 29, or 30, linked nucleosides. The first region and the second region of the core nucleic acid strand can have a same length or a different length. In some embodiments, the second region of the core nucleic acid strand has a relatively short length with respect to the first region of the core nucleic acid strand. In some embodiments, the second region of the core nucleic acid strand has about 14 linked nucleosides.

In some embodiments, the first region of the core nucleic acid strand comprises a sequence complementary to a target RNA. The length of the sequence complementary to a target RNA can vary in different embodiments. In some embodiments, the sequence complementary to a target RNA is 10-35 nucleotides in length. For example, the sequence complementary to a target RNA is 10, 11, 12, 13, 14, 15, 16, 17, 18, 19, 20, 21, 22, 23, 24, 25, 26, 27, 28, 29, 30, 31, 32, 33, 34, or 35, nucleotides in length. In some embodiments, the sequence complementary to a target RNA is 10-21 nucleotides in length.

The first region of the core nucleic acid strand comprises a sequence complementary to a passenger nucleic acid strand. The length of the sequence complementary to a passenger nucleic acid strand can vary in different embodiments. In some embodiments, the sequence complementary to a passenger nucleic acid strand is 17-22 nucleotides in length. For example, the sequence complementary to a passenger nucleic acid strand is 17, 18, 19, 20, 21, or 22 nucleotides in length. In some embodiments, the sequence of the core nucleic acid strand complementary to a passenger nucleic acid strand is about 21 nucleotides in length.

In some embodiments, the first region of the core nucleic acid strand is linked to the second region of the core nucleic acid strand via a connector. For example, the first region of the core nucleic acid strand is linked the second region of the core nucleic acid strand via a 5′ connector. In some embodiments, the core nucleic acid strand only comprises one connector (e.g. 5′ connector) and does not comprise a 3′ connector.

The 5′ connector can comprise a three-carbon linker (C₃ linker), a nucleotide, any modified nucleotide described herein, or any moiety that can resist exonuclease cleavage when the core nucleic acid strand is single-stranded (e.g. after displacement of the sensor nucleic acid strand from the core nucleic acid strand). For example, the 5′ connector can comprise a 2′-F nucleotide such as 2′-F-adenosine, 2′-F-guanosine, 2′-F-uridine, or 2′-F-cytidine. The 5′ connector can comprise a 2′-O-methyl nucleotide such as 2′-O-methyladenosine, 2′-O-methylguanosine, 2′-O-methyluridine, or 2′-O-methylcytidine. The 5′ connector can comprise a naturally occurring nucleotide such as cytidine, uridine, adenosine, or guanosine. The 5′ connector of the core nucleic acid strand can comprise a phosphodiester linkage (phosphodiester 5′ and 3′ connection) cleavable by an exonuclease when in a single-stranded form. The 5′ connector and/or the 3′ connector of the core nucleic acid strand can comprise any suitable moiety that can resist exonuclease cleavage when in single-stranded form. In some embodiments, the 5′ connector of the core nucleic acid strand comprises no linker molecule except for the normal phosphodiester linkage connecting two adjacent nucleosides (see, for example, the CASi design shown in FIGS. 1-2).

The 5′ connector can comprise or is, a C₃ 3-carbon linker, a nucleotide, a modified nucleotide (e.g. 2′-O-methyl nucleotide, 2′-F nucleotide), a nucleotide with a phosphodiester 5′ and 3′ connection cleavable by an exonuclease when in a single stranded form, or a combination thereof. In some embodiments, the 5′ connector can comprise or is a 2′-O-methyl nucleotide such as 2′-O-methyladenosine, 2′-O-methylguanosine, 2′-O-methyluridine, or 2′-O-methylcytidine. In some embodiments, the 5′ connector can comprise or is 2′-F nucleotide such as 2′-F-adenosine, 2′-F-guanosine, 2′-F-uridine, or 2′-F-cytidine.

In some embodiments, the 5′ connector of the core nucleic acid strand does not comprise or is not a C₃ 3-carbon linker. In some embodiments, it is advantageous not to have a C₃ 3-carbon linker as the 5′ connector.

In some embodiments, a nucleic acid complex not having a C₃ 3-carbon linker as the 5′ connector may exhibit higher RNA interfering activity. In some embodiments, not having a C₃ 3-carbon linker as the 5′ connector can increase RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these value, greater than nucleic acid complexes having a C3 3-carbon linker as the 5′ connector.

In some embodiments, the core nucleic acid strand do not comprise a 5′ connector. Instead, the first region of the core nucleic acid strand is linked to the second region via a standard phosphodiester linkage connecting two adjacent nucleosides.

In some embodiments, not having a 5′ connector between the first region and the second region of the core nucleic acid strand can increase RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or a number or a range between any of these value, greater than nucleic acid complexes having a linker (e.g. C₃ 3-carbon linker, a nucleotide, a modified nucleotide, or other moieties) as the 5′ connector.

In some embodiments, the core nucleic acid strand has an overhang. The overhang can be at the 3′ terminus of the core nucleic acid strand (3′ overhang). In some embodiments, the core nucleic acid strand can have a short overhang at the 3′ terminus (e.g. 1-3 nucleosides), but the 3′ overhang does not extend back into the middle of the sensor duplex to bind with the sensor nucleic acid strand (see, for example, FIGS. 1-2). The length of the overhang can vary in different embodiments. In some embodiments, the 3′ overhang is about one to three nucleotides in length. For example, the 3′ overhang can be one, two or three nucleotides in length. The overhang can comprise one or more modified nucleotides, such as 2′-O-methyl nucleotides. For example, the 3′ overhang can comprise two 2′-O-methyl nucleotides (see, for example, the CASi design shown in FIGS. 1-2). The overhang can comprise modified internucleoside linkages, such as phosphorothioate internucleoside linkages. In some embodiments, all of the nucleotides in the overhang are chemically modified. In some embodiments, all of internucleoside linkages in the 3′ overhang of the core nucleic acid strand are phosphorothioate internucleoside linkages.

### Passenger Nucleic Acid Strand

The passenger nucleic acid strand of the nucleic acid complex described herein is complementary bound to the first region of the core nucleic acid strand to form a RNAi duplex (e.g. a first nucleic acid duplex). In some embodiments, the first region of the core nucleic acid strand comprises a sequence complementary to a target nucleic acid strand, the passenger nucleic strand of the nucleic acid complex can comprise a sequence homologous to the target nuclei acid strand.

As used herein, the term “homologous” or “homology” refers to sequence identity between at least two sequences. The term “sequence identity” or “identity” in the context of two nucleic acid or polypeptide sequences makes reference to the nucleotide bases or residues in the two sequences that are the same when aligned for maximum correspondence over a specified comparison window.

In some embodiments, the sequence identity between a passenger nucleic acid strand and a target nucleic acid or a portion there of can be, be about, be at least, or be at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. The passenger nucleic acid strand of a nucleic acid complex can have a sequence substantially identical, e.g. at least 80%, 90%, or 100%, to a target nucleic acid or a portion thereof.

The length of the passenger nucleic acid strand can vary in different embodiments. In some embodiments, the passenger nucleic acid strand comprises 10-35 linked nucleosides. For example, the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the passenger nucleic acid strand comprises 17-21 linked nucleosides.

In some embodiments, the passenger nucleic acid strand has a 3′ overhang, a 5′ overhang, or both in the RNAi duplex. In some embodiments, the passenger nucleic acid strand has a 3′ overhang, and the 3′ overhang is one to five nucleosides in length.

In some embodiments, the overhang of the passenger nucleic acid strand is capable of binding to the input nucleic acid strand to form a toehold, thereby initiating a toehold mediated strand displacement and causing the displacement of the passenger nucleic acid strand from the core nucleic acid strand.

In some embodiments, the overhang of the passenger nucleic acid strand is 5 to 20 nucleosides in length. For example, the overhang of the passenger nucleic acid strand can be 5, 6, 7, 8, 9, 10, 11, 12, 13, 14, 15, 16, 17, 18, 19, or 20 nucleosides in length. In some embodiments, the overhang of the passenger nucleic acid strand is 9 nucleosides in length.

In some embodiments, one or more internucleoside linages of the overhang of the passenger nucleic acid strand are phosphorothioate internucleoside linkage which can protect the overhang from degradation. In some embodiments, all internucleoside linages of the overhang of the passenger nucleic acid strand can be phosphorothioate internucleoside linkage.

In some embodiments, the passenger nucleic acid strand is fully complementary o the first region of the core nucleic acid strand, thereby forming no overhang at the 5′ and 3′ termini of the passenger nucleic acid strand in the RNAi duplex. Therefore, in some embodiments, the passenger nucleic acid strand does not have a 3′ overhang, a 5′ overhang, or both in the RNAi duplex. In some embodiments, having a blunt end with no overhang can render the passenger nucleic acid strand unfavorable for Dicer binding, thereby bypassing the Dicer-mediated cleavage.

In some embodiments, the passenger nucleic acid strand is attached to a terminal moiety and/or a blocking moiety. Any suitable terminal moiety described herein that is capable of blocking the passenger nucleic acid strand from interacting with a RNAi pathway enzyme (e.g. Dicer, RISC) can be used. The blocking moiety can include one or more suitable terminal linkers or modifications such as a blocker that can protect a single-stranded nucleic acid from nuclease degradation such as an exonuclease blocking moiety. Examples of suitable blocking moieties include, but are not limited to, a dye (e.g. fluorophore, Cy3, a dark quencher), inverted dT, a linker to link the oligonucleotide with another molecule or a particular surface (biotins, amino-modifiers, alkynes, thiol modifiers, azide, N-Hydroxysuccinimide, and cholesterol), a space (e.g. C3 spacer, Spacer 9, Spacer 18, dSpacer, tri-ethylene glycol spacer, hexa-ethylene glycol spacer), a fatty acid, one or more modified nucleotides (e.g. 2′-O-methyl, 2′-F, PS backbone connection, LNA, and/or 2′-4′ bridged base) or a combination thereof. In some embodiments, the 5′ terminus of the passenger nucleic acid is attached to an inverted-dT, a tri-ethylene-glycol, or a fluorophore. For example, a fluorophore can be attached to the 5′ terminus of the passenger nucleic acid strand via a phosphorothioate linkage.

### Sensor Nucleic Acid Strand

The sensor nucleic acid strand of the nucleic acid complex described herein comprises a region complementary bound to the second region of the core nucleic acid strand to form a sensor duplex (e.g. a second nucleic acid duplex). The length of the region complementary bound to the second region of the core nucleic acid strand can vary in different embodiments. In some embodiments, the region complementary bound to the second region of the core nucleic acid strand comprises 10-35 linked nucleosides. For example, the region in the sensor nucleic acid strand complementary bound to the second region of the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the region in the sensor nucleic acid strand complementary bound to the second region of the core nucleic acid strand can comprise 10-30 linked nucleosides. In some embodiments, the region in the sensor nucleic acid strand complementary bound to the second region of the core nucleic acid strand comprise about 14 linked nucleosides.

The sensor nucleic acid strand doe not bind to the first region of the core nucleic acid strand nor any region 3′ of the first region of the core nucleic acid strand.

The sensor nucleic acid strand can comprise an overhang. The overhang can be at the 3′ end or 5′ end, or both, of the sensor nucleic acid strand. For example, the overhang can be at the 3′ of the region complementary bound to the second region of the core nucleic acid strand. The overhang is not complementary to the core nucleic acid strand and is capable of binding to an input nucleic acid strand, thereby initiating a toehold mediated strand displacement and causing the displacement of the sensor nucleic acid strand from the core nucleic acid strand.

The length of the overhang in the sensor nucleic acid strand can vary in different embodiments. In some embodiments, the length of the overhang can be 5-20 linked nucleotides. For example, the length of the overhang in the sensor nucleic acid strand can comprise 5, 6, 7, 8, 9, 10, 11, 12, 13, 14, 15, 16, 17, 18, 19, or 20 nucleotides in length. In some embodiments, the overhang of the sensor nucleic acid strand is 12 nucleotides in length.

The overhang of the sensor nucleic acid strand can comprise nucleotide modification introduced to improve the base-pairing affinity, nuclease resistance of the singled-stranded overhang, and thermodynamic stability to avoid spurious exonuclease induced activation of the strand. Exemplary modifications include, but not limited to, 2′-O-methyl modification, 2′-Fluoro modifications, phosphorothioate internucleoside linkages, inclusions of LNA, and the like that are identifiable by a skilled person. In some embodiments, at least 50% of the internucleoside linkages in the overhang of the sensor nucleic acid strand are phosphorothioate internucleoside linkages. For example, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, of the internucleoside linkages in the overhang of the sensor nucleic acid strand are phosphorothioate internucleoside linkages. In some embodiments, all internucleoside linkages in the overhang of the sensor nucleic acid strand are phosphorothioate internucleoside linkages.

The 5′ terminus and/or the 3′ terminus of the sensor nucleic acid strand can comprise a terminal moiety. Any suitable terminal moiety described herein can be used. In some embodiments, the terminal moiety can include a tri- or hexa-ethylene glycol spacer, a C3 spacer, an inverted dT, an amine linker, a ligand (e.g. a delivery ligand), a fluorophore, an exonuclease, a fatty acid, a Cy3, an inverted dT attached to a tri-ethylene glycol, or a combination thereof. In some embodiments, the 3′ terminus of the sensor nucleic acid strand can be attached to a delivery ligand, a dye (e.g. fluorophore), or exonuclease. The 5′ terminus can be attached to a fatty acid, a dye (e.g. Cy3), an inverted dT, a tri-ethylene glycol, or an inverted dT attached to a tri-ethylene glycol. The delivery ligand attached to the 3′ terminus can be any suitable ligand for use in targeting the nucleic acid complex to specific cell types described elsewhere in the present disclosure. In some embodiments, the delivery ligand is a palmitic acid. In some embodiments, the palmitic acid is attached to the 3′ terminus of the sensor nucleic acid strand. In some embodiments, a nucleic acid complex construct comprising a sensor nucleic acid strand with a palmitic acid attached to the 3′ terminus of the sensor nucleic acid can achieve a higher degree of inhibition of a target nucleic acid in comparison to corresponding constructs without a 3′ terminal palmitic acid.

The sequence of the sensor nucleic acid strand can be designed to sense an input nucleic acid strand or a portion thereof. For example, from the sequence of an input biomarker, a list of all possible sensor segments which are antisense to the input strand can be generated. The sensor sequences for uniqueness in the transcriptome of the target animal can be ranked using NCBI BLAST. For human cancer cell lines, sequences can be checked against human transcript and genomic collection using the BLASTn algorithm. In some embodiments, sensor segments that have more than 17 bases of sequence complementarity and complete overhang complementarity to known or predicted RNA transcripts may be eliminated. Examples of design features of the sensor nucleic acid strand that can be used in the nucleic acid complexes described herein are described, for example, in WO/2020/033938, the content of which is incorporated herein by reference.

### Input Nucleic Acid Strand

The input nucleic acid strand described herein acts as a trigger to activate (switch on) the RNA interfering activity of the nucleic acid complex (e.g. RNAi duplex) upon binding to a sequence of the sensor nucleic acid in the nucleic acid complex.

The input nucleic acid strand comprises a sequence complementary to a sequence in the sensor nucleic acid of the nucleic acid complex, such as, in the overhang region of the sensor nucleic acid. The complementary binding between the input nucleic acid strand and the sensor nucleic acid strand (e.g. an overhang) causes displacement of the sensor nucleic acid strand from the core nucleic acid strand, thereby activating the RNA interfering activity of the RNAi duplex formed by the passenger nucleic acid strand and the first region of the core nucleic acid strand.

The input nucleic acid strand can be cellular RNA transcripts that are present at relatively high expression levels in a set of target cells (e.g. cancer cells) and at a relatively low level of expression in a set of non-target cells (e.g. normal cells). In some embodiments, the nucleic acid complex herein described is activated (switched on) in target cells; whereas in the non-target cells, the nucleic acid complex remains inactivated (switched off).

In the target cells, the input nucleic acid strand can be expressed at a level of, about, at least, or at least about 2-fold, 5-fold, 10-fold, 20-fold, 30-fold, 40-fold, 50-fold, 60-fold, 70-fold, 80-fold, 90-fold, or 100-fold higher than in the non-target cells.

In the target cells, the input nucleic acid strand can be expressed at a level of, about, at least, at least about 50, 100, 200, 300, 400, 500, 600, 700, 800, 900, 1000 transcripts. In some embodiments, in the non-target cells, the input nucleic acid strand is expressed at a level of less than 50, less than 40, less than 30, less than 20, or less than 10 transcripts. Preferably, the non-target cells have no detectable expression of the input nucleic acid strand.

The input nucleic acid strand can comprise an mRNA, an miRNA, or a non-coding RNA such as a long non-coding RNA, an RNA fragment, or an RNA transcript of a virus. In some embodiments, the input nucleic acid strand is an RNA transcript that is expressed in a set of cells that are causing the progression of a disease and are therefore targeted for RNAi therapy. The non-target cells are usually a set of cells where silencing of a target RNA can cause side effects that are not beneficial for therapy. For treating a disease or a condition where the input RNA is overexpressed in target cells, the nucleic acid complex can be designed such that the sensor nucleic acid strand comprises a sequence complementary to the input RNA sequence. Upon administration of the nucleic acid complex, the binding of sensor nucleic acid strand to the input RNA induces the dissociation of the RNAi duplex from the sensor duplex in target cells thereby to activate the RNAi targeting the disease or condition.

In some embodiments, the input nucleic acid strand comprises a biomarker. The term “biomarker” refers to a nucleic acid sequence (DNA or RNA) that is an indicator of a disease or disorder, a susceptibility to a disease or disorder, and/or of response to therapeutic or other intervention. A biomarker can reflect an expression, function or regulation of a gene. The input nucleic acid strand can comprise any disease biomarker known in the art.

In some embodiments, the input nucleic acid strand is a mRNA, for example a cell type or cell state specific mRNA. Examples of a cell type or cell-state specific mRNA include, but are not limited to, C3, GFAP, NPPA, CSFIR, SLCIA2, PLP1, and MBP mRNA. In some embodiments, the input nucleic acid is a microRNA (also known as miRNA), including but is not limited to, hsa-mir-23a-3p, hsa-mir-124-3p, and hsa-mir-29b-3p. In some embodiments, the input nucleic acid strand is a non-coding RNA, for example MALAT1 (metastasis associated lung adenocarcinoma transcript 1, also known as NEAT2 (noncoding nuclear-enriched abundant transcript 2).

### Target RNA

The first region of the core nucleic acid strand can, for example, comprise a sequence complementary to a target RNA in order to direct target-specific RNA interference. In some embodiments, the target RNA is a cellular RNA transcript. The target RNA can be an mRNA, an miRNA, a non-coding RNA, a viral RNA transcript, or a combination thereof.

As used herein, a “target RNA” refers to a RNA whose expression is to be selectively inhibited or silenced through RNA interference. A target RNA can be a target gene comprising any cellular gene or gene fragment whose expression or activity is associated with a disease, a disorder or a condition. A target RNA can also be a foreign or exogenous RNA or RNA fragment whose expression or activity is associated with a disease, a disorder or a certain condition (e.g. a viral RNA transcript or a pro-viral gene).

The target RNA can comprise an oncogene, a cytokinin gene, an idiotype protein gene (Id protein gene), a prion gene, a gene that expresses a protein that induces angiogenesis, an adhesion molecule, a cell surface receptor, a gene of a protein involved in a metastasizing and/or invasive process, a gene of a proteinase, a gene of a protein that regulates apoptosis and the cell cycle, a gene that expresses the EGF receptor, a multi-drug resistance 1 gene (MDR1), a gene of a human papilloma virus, a hepatitis C virus, or a human immunodeficiency virus, a gene involved in cardiac hypertrophy, or a fragment thereof.

The target RNA can comprise a gene encoding for a protein involved in apoptosis. Exemplary target RNA genes include, but are not limited to, bcl-2, p53, caspases, cytotoxic cytokines such as TNF-α or Fas ligand, and a number of other genes known in the art as capable of mediating apoptosis. The target RNA can comprise a gene involved in cell growth. Exemplary target RNA genes include, but not limited to, oncogenes (e.g., genes encoding for ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES), as well as genes encoding for tumor suppressor proteins (e.g., APC, BRCA1, BRCA2, MADH4, MCC, NF I, NF2, RB I, TP53, and WTI). The target RNA can comprise a human major histocompatibility complex (MHC) gene or a fragment thereof. Exemplary MHC genes include MHC class I genes such as genes in the HLA-A, HLA-B or HLA-C subregions for class I cc chain genes, or B2-microglobulinand and MHC class II genes such as any of the genes of the DP, DQ and DR subregions of class II α chain and β chain genes (i.e. DPα, DPβ, DQα, DQβ, DRα, and DRβ).

The target RNA can comprise a gene encoding for a pathogen-associated protein. Pathogen associated protein include, but are not limited to, a viral protein involved in immunosuppression of the host, replication of the pathogen, transmission of the pathogen, or maintenance of the infection, or a host protein which facilitates entry of the pathogen into the host, drug metabolism by the pathogen or host, replication or integration of the pathogen's genome, establishment or spread of infection in the host, or assembly of the next generation of pathogen. The pathogen can be a virus, such as a herpesvirus (e.g., herpes simplex, varicella-zoster virus, Epstein-Barr virus, cytomegalovirus (CMV)), hepatitis C, HIV, JC virus), a bacteria or a yeast.

The target RNA can comprise a gene associated with a disease or a condition of the central nervous system (CNS). Exemplary genes associated with a CNS disease or a condition include, but are not limited to, APP, MAPT, SOD1, BACE1, CASP3, TGM2, NFE2L3, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, ATF6, CASP2, CASP1, CASP7, CASP8, CASP9, HRK, CIQBP, BNIP3, MAPK8, MAPK14, Rac1, GSK3B, P2RX7, TRPM2, PARG, CD38, STEAP4, BMP2, GJA1, TYROBP, CTGF, ANXA2, RHOA, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, and HSP47.

### Pharmaceutical Compositions and Methods of Administration

### Compositions

Also provided herein include pharmaceutical compositions comprising the nucleic acid complex as herein described, in combination with one or more compatible and pharmaceutically acceptable carriers.

The nucleic acid complex herein described can be suitably formulated and introduced into cell environment by any means that allows for a sufficient portion of the constructs to enter the cells to induce gene silencing, if it occurs.

The nucleic acid complex can be admixed, encapsulated, conjugated, or associated with other molecules, molecule structures, mixtures of compounds or agent, or other formulations for assistance in uptake, distribution, and/or absorption during delivery.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, pharmaceutically acceptable carrier comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

In some embodiments, pharmaceutically acceptable salts to be used with the nucleic acid complex herein described include but are not limited to (1) salts formed with cations such as sodium, potassium, ammonium, magnesium, calcium, polyamines such as spermine and spermidine, etc.; (2) acid addition salts formed with inorganic acids, for example hydrochloric acid, hydrobromic acid, sulfuric acid, phosphoric acid, nitric acid and the like; (3) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene disulfonic acid, polygalacturonic acid, and the like; and (4) salts formed from elemental anions such as chlorine, bromine, and iodine.

### Delivery Vesicles

Various delivery systems can be employed for delivering the nucleic acid complex herein described such as antibody conjugates, micelles, natural polysaccharides, peptides, synthetic cationic polymers, microparticles, lipid-based nanovectors among others.

Delivery systems and the related excipients used for delivery of the nucleic acid complex herein described can vary in different embodiments. Delivery systems can be selected based on the mode of administration utilized, types of formulations, target sites, and types of diseases or disorders to be treated to facilitate tissue penetration, cellular uptake and to prevent extravasation and endosomal escape.

In some embodiments, the nucleic acid complex can be formulated with one or more polymers to form a supramolecular complex containing the nucleic acid complex and a multi-dimensional polymer network. The polymer can be linear or branched. The supramolecular complex can take any suitable form, and preferably, is in the form of particles.

The nucleic acid complex can be delivered via a lipid-mediated delivery system. In some embodiments, the nucleic acid complex can be encapsulated or associated with liposomes. For example, the nucleic acid complex can be condensed with a polycationic condensing agent, suspended in a low-ionic strength aqueous medium and cationic liposomes formed of a cationic vesicle-forming lipid.

As used herein, the term “liposomes” refers to lipid vesicles having an outer lipid shell, typically formed on one or more lipid bilayers, encapsulating an aqueous interior. In some embodiments, the liposomes are cationic liposomes composed of between about 20-80 mole percent of a cationic vesicle-forming lipid, with the remaining neutral vesicle-forming lipids and/or other components. As used herein, “vesicle-forming lipid” refers to any amphipathic lipid having hydrophobic and polar head group moieties and which by itself can form spontaneously into bilayer vesicles in water (e.g. phospholipids). A preferred vesicle-forming lipid is a diacyl-chain lipid, such as a phospholipid, whose acyl chains are typically between about 14-22 carbon atoms in length, and have varying degrees of unsaturation.

A cationic vesicle-forming lipid is a vesicle-forming lipid whose polar head group with a net positive charge, at the operational pH, e.g., pH 4-9. Examples include phospholipids (e.g. phosphatidylethanolamine), glycolipids (e.g. cerebrosides and gangliosides having a cationic polar head-group), cholesterol amine and related cationic sterols (e.g. 1,2-diolelyloxy-3-(trimethylanuno) propane (DOTAP), N-[1-(2,3,-ditetradecyloxy) propyl]-N,N-dimethyl-N-hydroxyethylammonium bromide (DMRIE), N-[1-(2,3,-dioleyloxy) propyl]-N,N-dimethyl-N-hydroxy ethylammonium bromide (DORIE), N-[1-(2,3-dioleyloxy) propyl]-N,N,N-trimethylammonium chloride (DOTMA), 3β [N—(N′,N′-dimethylaminoethane) carbamoyl] cholesterol (DC-Choi), and dimethyldioctadecylammonium (DDAB)).

A neutral vesicle-forming lipid is a vesicle-forming lipid having no net charge or including a small percentage of lipids having a negative charge in the polar head group. Examples of vesicle-forming lipids include phospholipids, such as phosphatidylcholine (PC), phosphatidyl ethanolamine (PE), phosphatidylinositol (PI), and sphingomyelin (SM), and cholesterol, cholesterol derivatives, and other uncharged sterols.

In some embodiments, the delivery systems used herein include, but are not limited to, nanoparticles (NPs), inorganic nanoparticles (e.g. slica NPs, gold NPs, Qdots, superparamagnetic iron oxide NPs, paramagnetic lanthanide ions) and other nanomaterials, nucleic acid lipid particles, polymeric nanoparticles, lipidoid nanoparticles (LNPs), chitosan and inulin nanoparticles, cyclodextrins nanoparticles, carbon nanotubes, liposomes, micellar structures, capsids, polymers (e.g. polyethylenimine, anionic polymers), polymer matrices, hydrogels, dendrimers (e.g. poly-propylenimine (PPI) and poly-amidoamine (PAMAM)), nucleic acid nanostructure, exosomes, and GalNAc-conjugated melittin-like peptides (NAG-MLPs). In some embodiments, the nucleic acid complex can be formulated in buffer solutions such as phosphate buffered saline solutions.

In some embodiments, the nucleic acid complex herein described is delivered via lipidoid nanoparticles (LNPs). LNPs can comprise ionizable LNPs, cationic LNPs, and/or neutral LNPs. Ionizable LNPs are nearly uncharged during circulation but become protonated in a low pH environment, e.g., in the endosomes and lysosomes. Cationic LNPs exhibit a constitutive positive charge in blood circulation and in endosomes or lysosomes. Neutral LNPs are neutral, uncharged during circulation and in endosomes or lysosomes.

The nucleic acid complex herein described can be provided naked or conjugated to a ligand. Naked siRNA refer to a system that contains no delivery system that is associated with the siRNA either covalently or noncovalently. When delivered in naked form, the naked siRNAs can be locally injected to a target site such as specific organs that are relatively closed off and contain few nucleases (e.g. eye).

In some embodiments, the nucleic acid complex herein described can be attached to (e.g. fused or conjugated) a ligand to form ligand-siRNA conjugates that can transport siRNA to desired tissues and cells by specific recognition and interactions between the ligand and the surface receptor of the cells or tissues. Common targeting ligands include carbohydrate, aptamers, antibodies or antibody fragments, peptides (e.g. cell-penetrating peptides, endosomolytic peptides), and small molecules (e.g. N-Acetylgalactosamine (GalNAc)), and others as will be apparent to a skilled artisan.

In some embodiments, the nucleic acid complex is conjugated to an aptamer. The term “aptamers” as used here refers to oligonucleotide or peptide molecules that bind a specific target with high affinity and specificity. In particular, nucleic acid aptamers can comprise, for example, nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are peptides that are designed to specifically bind to and interfere with protein-protein interactions inside cells. In particular, peptide aptamers can be derived, for example, according to a selection strategy that is derived from the yeast two-hybrid (Y2H) system. Aptamers are useful in biotechnological and therapeutic applications as they offer molecular recognition properties that rival that of the antibodies.

In some embodiments, the nucleic acid complex is conjugated to a small molecule. The term “small molecule” as used herein indicates an organic compound that is of synthetic or biological origin and that, although may include monomers and/or primary metabolites, is not a polymer. In some embodiments, small molecules can comprise molecules that are not protein or nucleic acids, which play a biological role that is endogenous (e.g. inhibition or activation of a target) or exogenous (e.g. cell signaling), which are used as a tool in molecular biology, or which are suitable as drugs in medicine. Small molecules can also have no relationship to natural biological molecules. Typically, small molecules have a molar mass lower than 1 kg/mol. Exemplary small molecules include secondary metabolites (such as actinomicyn-D), certain antiviral drugs (such as amantadine and rimantadine), teratogens and carcinogens (such as phorbol 12-myristate 13-acetate), natural products (such as penicillin, morphine and paclitaxel) and additional molecules identifiable by a skilled artisan. In some embodiments, the nucleic acid complex herein described is conjugated to GalNAc.

Examples of ligands suitable for use in targeting the nucleic acid complex to specific cell types include, but are not limited to, folate capable of binding to folate receptor of epithelial carcinomas and bone marrow stem cells, water soluble vitamins capable of binding to vitamin receptors of various cells, pyridoxal phosphate capable of binding to CD4 of CD4 lymphocytes, apolipoproteins capable of binding to LDL of liver hepatocytes and vascular endothelial cells, insulin capable of binding to insulin receptor, transferrin capable of binding to transferrin receptor of endothelial cells, galactose capable of binding to asialoglycoprotein receptor of liver hepatocytes, sialyl-Lewisx capable of binding to E, P selectin of activated endothelial cells, Mac-1 capable of binding to L selectin of neutrophils and leukocytes, VEGF capable of binding to Flk-1,2 of tumor epithelial cells, basic FGF capable of binding to FGF receptor of tumor epithelial cells, EFG capable of binding to EFG receptor of epithelial cells, VCAM-1 capable of binding to abi integrin of vascular endothelial cells, ICAM-1 capable of binding to anb2 integrin of vascular endothelial cells, PECAM-1/CD31 capable of binding to avb3 integrin of vascular endothelial cells and activated platelets, osteopontin capable of binding to arbi integrin and aᵥb₅ integrin of endothelial cells and smooth muscle cells in atherosclerotic plaques, RGD sequences capable of binding to avb3 integrin of tumor endothelial cells and vascular smooth muscle cells, or HIV GP 120/41 or GP120 capable of binding to CD4 of CD4 lymphocytes, and others identifiable to a skilled artisan.

In some embodiments, the delivery of the nucleic acid complex herein described is such that at least 30%, 40%, 50%, 60%, 70%, 80%, 90% or more of the target cells incorporate the nucleic acid complex. In some embodiments, about 0.1-10 nm nucleic acid complex is delivered to the target cells.

### Formulations

Any suitable pharmaceutical formulations can be employed. In some embodiments, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the RNAi constructs which produces a therapeutic effect. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A nucleic acid complex composition may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Actual dosage levels of the active ingredients in the pharmaceutical compositions of the present disclosure may be determined by the methods of the present invention so as to obtain an amount of the active ingredient, which is effective to achieve the desired therapeutic response for a particular subject, composition, and mode of administration, without being toxic to the subject.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of hydrogel compositions include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions (e.g. eye drops) and the like, are also contemplated as being within the scope of the present disclosure.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions herein described comprise a therapeutically-effective amount of the nucleic acid complexes.

The phrase “therapeutically-effective amount” as used herein means that amount of nucleic acid complex disclosed herein which is effective for producing some desired therapeutic effect, e.g., cancer treatment, at a reasonable benefit/risk ratio. The therapeutically-effective amount also varies depending on the structure of the constructs, the route of administration utilized, the target sites, and the specific diseases or disorders to be treated as will be understood to a person skilled in the art. For example, if a given clinical treatment is considered effective when there is at least a 20% reduction in a measurable parameter associated with a disease or disorder, a therapeutically-effective amount of the constructs for the treatment of that disease or disorder is the amount necessary to achieve at least a 20% reduction in that measurable parameter.

In some embodiments, the pharmaceutical composition herein described comprises the nucleic acid complex in a suitable dosage sufficient to inhibit expression of the target gene in a subject (e.g. animal or human) being treated. In some embodiments, a suitable dosage of the nucleic acid complex is in the range of 0.001 to 0.25 milligrams per kilogram body weight of the subject per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical compositions comprising the nucleic acid complex can be administered once daily, twice daily, three times daily or as needed or prescribed by a physician. The pharmaceutical composition herein described can also be provided in dosage units comprising two, three, four, five, six or more sub-doses administered at appropriate intervals throughout the day. The dosage unit can also be compounded for a single dose (e.g. using sustained or controlled release formulation) which can be sustainably released over several days in a controlled manner.

As will be apparent to a skilled person, a suitable dosage unit of the pharmaceutical composition herein described can be estimated from data obtained from cell culture assays and further determined from data obtained in animal studies. For example, toxicity and therapeutic efficacy of the pharmaceutical compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. Suitable dosages of the compositions in combination with particular delivery systems can be selected in order to minimize toxicity, such as to minimize potential damage to untargeted cells and to reduce side effects.

### Administration

As will be apparent to a skilled artisan, the nucleic acid complexes herein described and compositions thereof can be administrated to a subject using any suitable administration routes. The nucleic acid complexes and compositions thereof can be administered to a target site locally or systematically.

The wording “local administration” or “topic administration” as used herein indicates any route of administration by which a composition is brought in contact with the body of the individual, so that the resulting composition location in the body is topic (limited to a specific tissue, organ or other body part where the imaging is desired). Exemplary local administration routes include injection into a particular tissue by a needle, gavage into the gastrointestinal tract, and spreading a solution containing hydrogel composition on a skin surface.

The wording “systemic administration” as used herein indicates any route of administration by which a nucleic acid complex composition is brought in contact with the body of the individual, so that the resulting composition location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In some embodiments, the methods of administration can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous (IV) injection, subcutaneous (SC) injection, intranodal injection, intratumoral injection, intraperitoneal injection, and/or intradermal injection, or any combination thereof. The administration can also be site-specific injection (e.g. in the eye or the cerebral spinal fluid).

In some embodiments, the administration can be Ex vivo transduction, cell injection, subcutaneous injection, intravenous injection, intrathecal delivery, intracerebroventricular injection, intradermal injection, intravitreal delivery, intratumoral delivery, or topical application (e.g. topical eye drop).

The methods of administration depends on the target site, the type of cells/tissues to be targeted at, and how the constructs are formulated. In some embodiments, lipid formulations can be administered to animals such as by intravenous, intramuscular, or intraperitoneal injection, or orally or by inhalation or other methods as known in the art.

In some embodiments, the administration can be SC injection into the adipose tissue below the epidermis and dermis. In some embodiments, SC administration can be associated with ligand-conjugated nucleic acid complex herein described. In some embodiments, SC administration can render a slower release rate of the drugs into the systemic circulation and an entering into the lymphatic system, giving more time for recycling of cellular receptors that mediate uptake. In some embodiments, SC administration can be faster and easier to administer, reducing treatment burden.

In some embodiments, the administration can be any administration route allowing the penetration of drugs through the blood brain barrier. In some embodiments, the route of administration can be direct brain injection, transmembrane diffusion, or intraventricular infusion of therapeutic substances directly into the cerebrospinal fluid. In some embodiments, the administration can be intrastriatal injection, intrathecal injection, intracerebral injection, intraparenchymal injection, intranasal delivery or intracerebroventricular injection. In some embodiments, the administration can be intracerebroventricular injection into the CNS to bypass the blood-brain barrier and other mechanisms that limit drug distribution to the brain, allowing a higher drug concentration to enter the central compartment.

In some embodiment, IV administration can be associated with nanoparticle and lipid nanoparticle formulated nucleic acid complex herein described. In some embodiments, IV administration can avoid first-pass metabolism in the liver and affords quick access to target tissue through the systemic circulation.

### Target Sites

The compositions herein described can be administered to any suitable target site. Target sites can be in vitro, in vivo or ex vivo. Exemplary target sites can include cells grown in an in vitro culture, including, primary mammalian, cells, immortalized cell lines, tumor cells, stem cells, and the like. Additional exemplary target sites include cells, tissues and organs in an ex vivo culture and cells, tissues, organs, or organs systems in vivo in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person.

In some embodiments, the target site is the central nervous system (e.g., brain and spinal cord), peripheral nervous system (e.g., nerves that branch off from the spinal cord) and connective tissues/organs involving in the function and pathways between the central and peripheral nervous systems (e.g., dorsal root and ventral root). The target site can include the brain, spinal cord, cranial nerves, peripheral nerves, nerve roots, autonomic nervous system, neuromuscular junction and muscles. In some embodiments, the target site is the central nervous system.

The target site can comprise a site of disease or disorder or can be proximate to a site of a disease or disorder. The location of the one or more sites of a disease or disorder can be predetermined. The location of the one or more sites of a disease or disorder can be determined during the method (e.g., by an imaging-based method such as ultrasound or MRI). The target site can comprise a tissue, such as, for example, adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can be inflamed tissue. The tissue can comprise (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, upon administration, the nucleic acid complex and a composition thereof accumulates in vasculature of cancerous tissue. In some embodiments, the target site can comprise a solid tumor. In some embodiments, the target site can comprise a tissue, such as, for example, grey matter, white matter, ganglion, nerves, endoneurium, perineurium, epineurium.

In some embodiments, target sites where the nucleic acid complex or compositions thereof can be administered can vary in different embodiments depending on the mode of administration utilized and the types of diseases or disordered to be treated. In some embodiments, the target sites can be related to ocular tissues, respiratory system, muscle, liver, central nerve system, solid tumors, hematopoietic system, skin, eye, placenta, bone, or other target sites in an individual as will be apparent to a skilled artisan.

The term “individual” or “subject” or “patient” as used herein in the context of imaging includes an animal and in particular higher animals and in particular vertebrates such as mammals and more particularly human beings.

In some embodiments, the ratio of the concentration of the nucleic acid complex at the subject's target site to the concentration of the nucleic acid complex outside the target site (e.g., in a subject's blood circulation, serum, or plasma) can vary. In some embodiments, the ratio of the concentration of the nucleic acid complex at the subject's target site to the concentration of the nucleic acid complex outside the target site (e.g. in subject's blood circulation, serum, or plasma) can be, or be about, be at least, be at least about, be at most, or be at most about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

The target site can comprise target cells. The target cells can be tumor cells (e.g., solid tumor cells). In some embodiments, the administration of the nucleic acid complex and/or compositions herein described to a target site of the subject results in the death of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the target cells. The ratio of target cell death to non-target cell death after administration of the nucleic acid complex and/or compositions can be at least about 2:1. In some embodiments, the ratio of target cell death to non-target cell death after administration of the nucleic acid complex and/or compositions can be, or be about, or be at least, or be at least about, or be at most, or be at most about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

In some embodiments, the target cells can include nerve cells and glial cells, including pyramidal cells, purkinje cells, granule cells, spindle neurons, nedium spiny neurons, interneurons, astrocyte, ependymal cells, microglia, oligodendrocyte, and oligodendrocyte progenitor cells. The target cells can also include dorsal root ganglion, ventral root ganglion, and automonic ganglion. In some embodiments, the administration of the nucleic acid complex and/or compositions herein described to a target site of the subject results in at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, reduction in the target nucleic acid expression in the target cells. In some embodiments, the ratio of reduction in the target nucleic acid in the target cells to non-target cell after administration of the nucleic acid complex and/or compositions can be at least about 2:1. In some embodiments, the ratio can be, or be about, or be at least, or be at least about, or be at most, or be at most about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

### Methods of Modulating a Target RNA

Also provided herein is a method of modulating a target RNA using the nucleic acid complex or a composition thereof herein described. The method can comprise contacting a cell comprising a target RNA with the nucleic acid complex herein describe. Upon detection of an input nucleic acid strand, an input strand can bind to the overhang of the sensor nucleic acid strand to cause displacement of the sensor nucleic acid strand from the core nucleic acid strand to release the sequence complementary to the target RNA into the cell, thereby modulating the target RNA.

Contacting the cells with the nucleic acid complex can be performed with cells in vitro, in vivo or ex vivo. For example, the cells can be cells grown in an in vitro culture, including, primary mammalian, cells, immortalized cell lines, tumor cells, stem cells, and the like. The cells can comprise cells, tissues and organs in an ex vivo culture and cells, tissues, organs, or organs systems in vivo in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The cell can be a disease cell or a cell of disorder. The cell can be a cancer cell. Contacting the cell with the nucleic acid complex can occur can also occur in vitro, ex vivo, or in vivo (e.g., in the body of a subject).

### Methods of Treating a Disease or Disorder

Also provided herein is a method of treating a disease or a condition using the nucleic acid complex or a composition thereof herein described. The method can comprise administering the nucleic acid complex described herein to a subject in need thereof. Upon detection of an input nucleic acid strand, the input nucleic acid strand can bind to the overhang of the sensor nucleic acid strand to cause displacement of the sensor nucleic acid strand from the core nucleic acid strand to release the sequence complementary to a target RNA, thereby reducing the activity of the target RNA or protein expression from the target RNA in the subject to treat the disease or condition.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

As used herein, the term “treatment” “treat” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The target RNA can comprise any gene described herein or known in the art whose expression or activity is associated with a disease or disorder (e.g., a neurological disease or cancer). For example, in some embodiments, the neurological disease is Huntington's disease and the target RNA comprises a HTT gene.

The sensor nucleic acid strand of the nucleic acid complex can be designed to detect any biomarker described herein or known in the pertinent art which is related to a disease or disorder such as a disease or disorder of the central nervous system or cancer. The biomarker can be a universal mRNA that is not cell type specific or selective (e.g., mir-23a-3p). The biomarker can also be a cell type or cell-state specific or selective mRNA such as mRNAs specific for cells of the central nervous system (e.g., GFAP mRNA).

In some embodiments, the nucleic acid complex is administered to the subject in need thereof at a concentration about 0.001-10 nM. For example, the nucleic acid complex can be provided at a concentration of, about, at most, or at most about, 0.001 nM, 0.002 mM, 0.004 mM, 0.006 mM, 0.008 mM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, or a number or a range between any two of these values. In some embodiments, the nucleic acid complex is provided at a concentration about 0.004-1.0 nM.

In some embodiments, the nucleic acid complex is administered to the subject in need thereof at a dosage about 1-100 mg/kg body weight of the subject, preferably 10-50 mg/kg body weight of the subject. Alternatively dosages may be based and calculated based upon the subject being treated, the severity and responsiveness of the condition to be treated, the manner of administration, and the judgement of the prescribing physician, as understood by those of skill in the art.

In some embodiments, the subject can be administered with the nucleic acid complex one, two, three, four or more times for the treatment. In some embodiments, at most one, two, three or four administrations are needed to achieve a desired treatment outcome. In some embodiments, only one administration is needed. Two administrations of the nucleic acid complex can be separated by a suitable time period. The suitable time period between two administrations can be the same as or different from the suitable time period between another two administrations. In some embodiments, the time period between two administrations can be about, at least or at least about 1 month, 2 months, 3 months, 4 months, 5 months, 6 months, 7 months, 8 months, 9 months, 10 months, 11 months, 12 months or longer. In some embodiments, the time period between any two administrations can be at least 6 months.

Various diseases and disorders can be treated with any one or more of the nucleic acid complex compositions provided herein. Diseases and disorders disclosed herein include, but are not limited to, HIV infection with lymphoma, hemophilia A, hemophilia B, hypercholesterolemia, atherosclerotic cardiovascular disease, renal impairment, chronic hepatitis B, acute intermittent porphyria, atypical hemolytic uraemic syndrome, primary hyperoxaluria, hereditary transthyretin amyloidosis (hATTR), α1-antitrypsin deficiency liver disease, hepatitis B, sickle cell disease, primary hyperoxaluria, ewing sarcoma, advanced gynecological cancer, stage III/IV ovarian cancer, pancreatic cancer, advanced solid tumors, hepatocellular carcinoma/liver cancer, lymphoma and leukemias, heart disease, heart failure, keloids, hypertrophic cicatrix, relapsed or refractory B cell lymphoma, hypertrophic scar, age-related macular degeneration, retinal scarring, cardia surgery, cardiac hypertrophy, non-arteritic anterior ischaemic optic neuropathy, alport syndrome, HIV infections/AIDS, pancreatic ductal adenocarcinoma/pancreatic cancer, dry eye disease, and various solid tumors.

In some embodiments, the disease or disorder can be a cancer. The cancer can be a solid tumor, a liquid tumor, or a combination thereof. The nucleic acid complex herein described or a composition thereof can be administered to the cells, tissues and/or organs comprising a tumor using any suitable administration route. For example, the nucleic acid complex or a composition thereof can be administered to the cells, tissues and/or organs comprising a tumor via subcutaneous injection or intratumoral delivery.

The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

The cancer can be a hematologic cancer, for example, chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

Non-limiting examples of cancers that can be prevented and/or treated using the nucleic acid complexes and compositions disclosed herein include: renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

In some embodiments, the disease or disorder can be a neurological disease or disorder. Neurological diseases or disorders are diseases or disorders of the central and peripheral nervous system including the brain, spinal cord, cranial nerves, peripheral nerves, nerve roots, autonomic nervous system, neuromuscular junction, and muscles. Neurological disorders can include epilepsy, Alzheimer's disease and other dementias, cerebrovascular diseases including stroke, migraine and other headache disorders, multiple sclerosis, Parkinson's disease, neuroinfections, brain tumors, and traumatic disorders of the nervous system due to head trauma.

In some embodiments, a disease or a disorder is a neurodegenerative disease or disorder. Neurodegenerative diseases or disorders are a heterogeneous group of disorders that are characterized by the progressive degeneration of the structure and function of the central nervous system or peripheral nervous system. In some embodiments, neurodegenerative diseases are diseases marked by continuous and progressive deterioration of the function of neural cells which are not caused by any underlying trauma or infection Exemplary neurodegenerative diseases or disorders include, but are not limited to, Alzheimer's disease (AD), Huntington's disease (HD), Parkinson's disease (PD), amyotrophic lateral sclerosis (ALS), and prion diseases.

In some embodiments, a disease or a disorder is a disease or condition of the central nervous system (CNS). Exemplary disease or a condition of the CNS include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

In some embodiments, the CNS disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a psychiatric disease, a neuroinflammatory disease, Alzheimer's disease, Huntington's disease, Parkinson's disease, Tourette syndrome, dystonia, or a combination thereof. In some embodiments, the disease is a neuroinflammatory disease. For example, the neuroinflammatory disease is Parkinson's disease, Alzheimer's disease, multiple sclerosis, or a combination thereof.

In some embodiments, the disease or disorder can be a central nervous system (CNS) or peripheral nervous system (PNS) disease or condition. The nucleic acid complex herein described or a composition thereof can be administered to the cells, tissues and/or organs of the CNS and/or PNS using any suitable administration route. For example, the nucleic acid complex or a composition thereof can be administered to the cells, tissues and/or organs of the CNS and/or PNS of a subject via intrathecal injection, intracerebroventricular injection, or intracerebral injection to penetrate the blood-brain barrier. In some embodiments, the cell(s), tissue(s), and/or organ(s) of the CNS and/or PNS comprises damaged or inflamed cell(s), tissue(s), or organ(s). In some embodiments, the cells(s), tissue(s), and/or organ(s) of the CNS and/or PNS comprise the brain, the white matter, the gray matter, the brainstem, the cerebellum, the diencephalon, the cerebrum, the spinal cord, the cranial nerve, dorsal root ganglia, cell(s) of any of the preceding, tissue(s) of any of the preceding, or a combination thereof.

In some embodiments, the method herein described comprises administering a nucleic acid complex herein described to a subject in need thereof, allowing the nucleic acid complex to be distributed into one or more regions of the nervous system, thereby reducing the activity of the target RNA or protein expression from the target RNA in the one or more regions of the nervous system of the subject to treat the neurological disease or disorder. In some embodiments, administration of the nucleic acid complex allows the distribution of the nucleic acid complex to the one or more regions of the nervous systems comprising a central nervous system, a peripheral nervous system, or both.

In some embodiments, administration of the nucleic acid complex allows the distribution of the nucleic acid complex to one or more regions of the nervous system comprising connective tissues/organs involving in the function and pathways between the central and peripheral nervous systems. In some embodiments, the one or more regions of the nervous system comprises dorsal root ganglion that carries sensory neural signals to the CNS from the PNS.

In some embodiments, administration of the nucleic acid complex allows the distribution of the nucleic acid complex to one or more regions of the central nervous system. The one or more regions of the central nervous system can comprise the brain, the white matter, the gray matter, the brainstem, the cerebellum, the diencephalon, the cerebrum, the spinal cord, the cranial nerve, or a combination thereof. In some embodiments, the one or more regions of the central nervous system comprise spinal cord, cerebrum (e.g., frontal lobe, parietal lobe, occipital lobe, temporal lobe, left hemisphere, and right hemisphere), cerebral cortex (e.g., prefrontal cortex, sensory cortex, visual cortex, auditory cortex, motor cortex), basal ganglia (e.g., striatum), thalamus, subthalamus, epithalamus, hypothalamus, amygdala, hippocampus (e.g., ventral hippocampus, dorsal hippocampus, and intermediate hippocampus), cerebellum, brain stem (e.g., midbrain, pons, medulla oblongata), left hemisphere, right hemisphere, corpus callosum, or a combination thereof. In some embodiments, the one or more regions of the CNS comprises cerebral cortex, subicular cortex, hippocampus, corpus callosum, fornix, lateral ventricle, stria terminalis, caudate putamen, internal capsule, piriform cortex, globus pallidus, optic tract, amygdala, anterior commissure, ventral striatum, lateral olfactory tract, cerebellum, pons, medulla, middle cerebellar peduncle, or a combination thereof.

In some embodiments, the administration of the nucleic acid complex to a subject in need thereof results in reduction or loss of expression of the target nucleic acid in the target cells. In some embodiments, the reduction of the target nucleic acid after the administration of the nucleic acid complex herein described is about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, with respect to the level of target nucleic acid prior to the administration. In some embodiments, the reduction occurs in one or more of the regions selected from the group consisting of: right cortex, prefrontal cortex, sensory cortex, visual cortex, striatum, dorsal hippocampus, ventral hippocampus, thalamus, cerebellum, midbrain, left hemisphere, right hemisphere, spinal cord upper, spinal cord lower, dorsal root ganglia, or a combination thereof.

In some embodiments, the nucleic acid complex described herein is specific and potent while achieving safety and tolerability in the subject being treated. For example, the administration of the nucleic acid complex does not result in a significant increase or decrease (e.g., plus or minus 5% of a base value or the difference is not statistically significant) in the body weight, inflammatory markers, blood chemistry, and/or liver, kidney pancreas enzymes in the subject with respect to the levels prior to the administration. In some embodiments, the administration of the nucleic acid complex does not induce unintended inflammatory responses. For example, in some embodiments, the administration the nucleic acid complex does not result an elevated glial fibrillary acidic protein (GFAP) and/or ionized calcium-binding adapter molecule 1 (IBA-1) level of the subject with respect to the levels prior to the administration. Kits

Also provided herein include kits comprising one or more compositions described herein, in suitable packaging such as in a container, pack, or dispenser, and may further comprise written material that can include instructions for use, discussion of clinical studies, listing of side effects, and the like. Such kits can also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. The compositions can be in the form of kits of parts. In a kit of parts, one or more components of the compositions disclosed herein are provided independent of one another (e.g., constructs, excipients, and/or diluents are provided as separate compositions) and are then employed (e.g., by a user) to generate the compositions.

### EXAMPLES

Some aspects of the embodiments discussed above are disclosed in further detail in the following examples, which are not in any way intended to limit the scope of the present disclosure.

### Example 1

### Determination of RNAi Activity

This example describes performing RNAi activity of various nucleic acid complex constructs described herein.

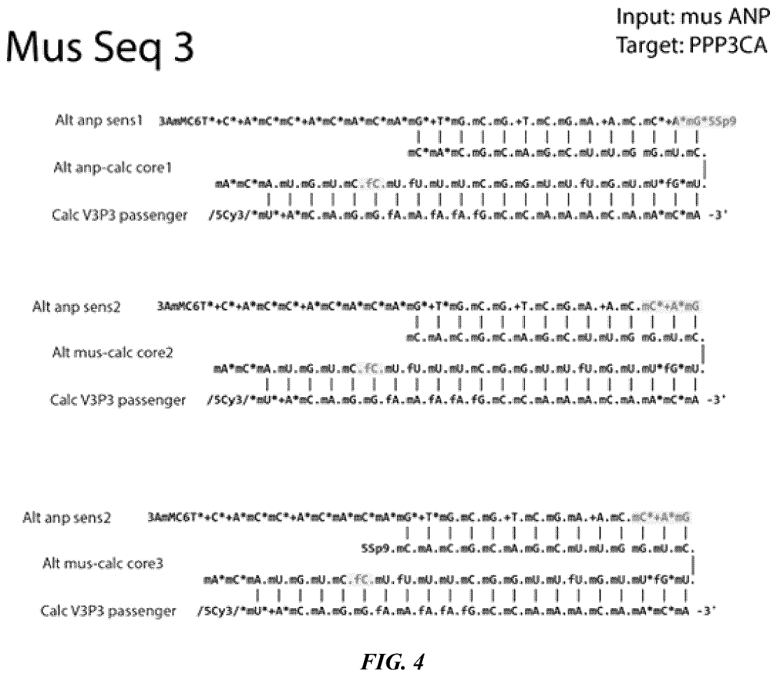

Different variants of the CASi siRNA constructs shown in FIG. 4 can be tested for RNAi activity. The sensor strand of the constructs can be designed to sense an input nucleic acid, such as a NPPA gene sequence encoding atrial natriuretic peptide (ANP). To test the constructs, CASi siRNA constructs can be assembled by thermally annealing the passenger strand, the core strand and the sensor strand in 1× phosphate buffer saline. The RNAi activities of the CASi siRNA constructs can be measured using dual luciferase assays. CASi siRNA constructs can be co-transfected into HCT 116 cells with dual luciferase vectors carrying a calcineurin gene target sequence (PPP3A), using lipofectamine 2000. After 48 hours, cells can be lysed and assayed for knockdown of the target gene by comparing the luminescence value of Renilla luciferase that carries the target sequence to Firefly luciferase that can be used as a reference control. Examples of methods and procedures of assembling CASi siRNA constructs, cell transfection, and dual luciferase assays are described in, for example, international application WO/2020/033938, the content of which is incorporated herein by reference in its entirety. It is expected that the RNA complexes described herein have RNAi activities.

### Example 2

### Implementation of an Exemplary T1 Conditionally Activated siRNA (TI CASi)

This example describes the implementation of a T1 CASi construct and further demonstrates the RNAi activity of the T1 CASi construct in the central and peripheral nervous system.

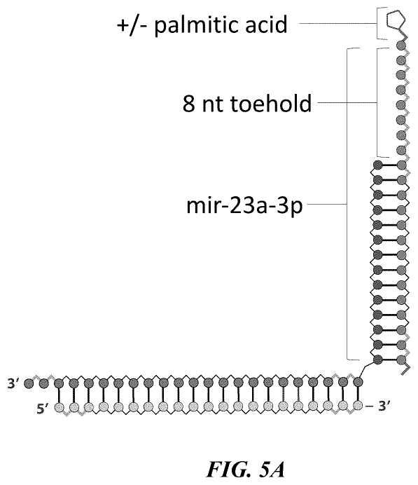

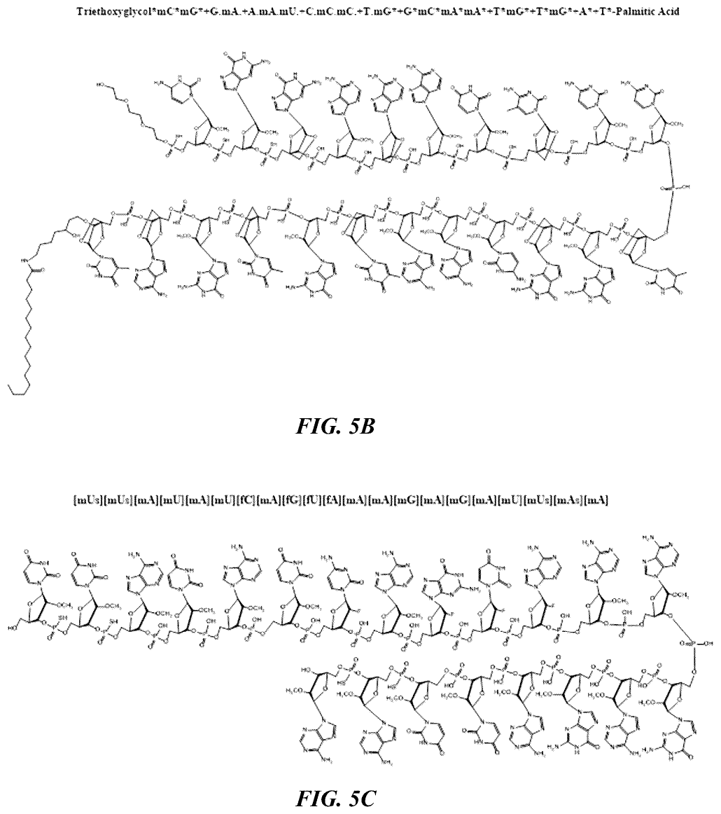

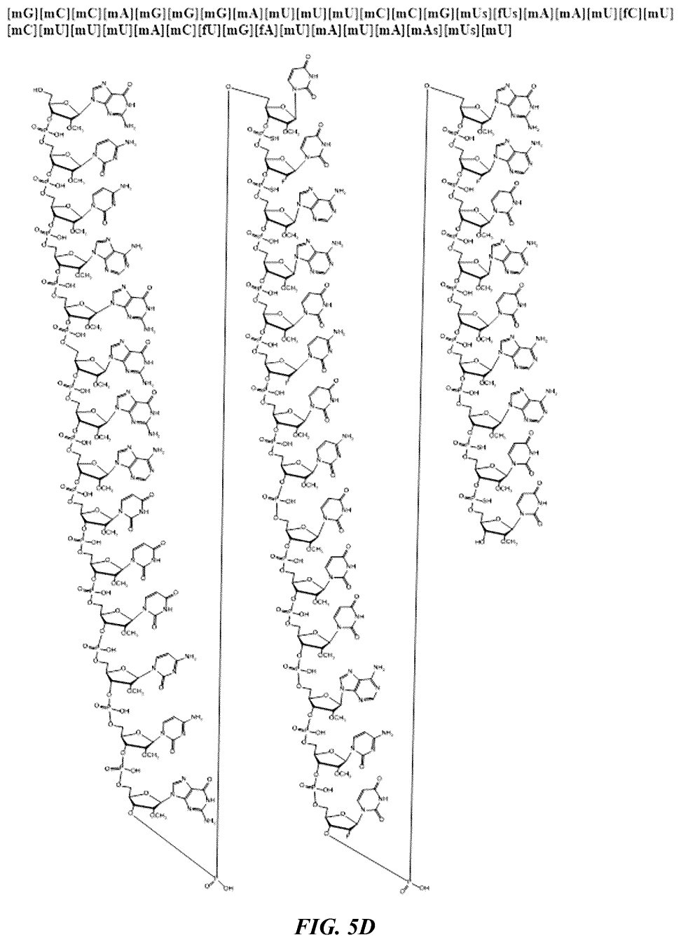

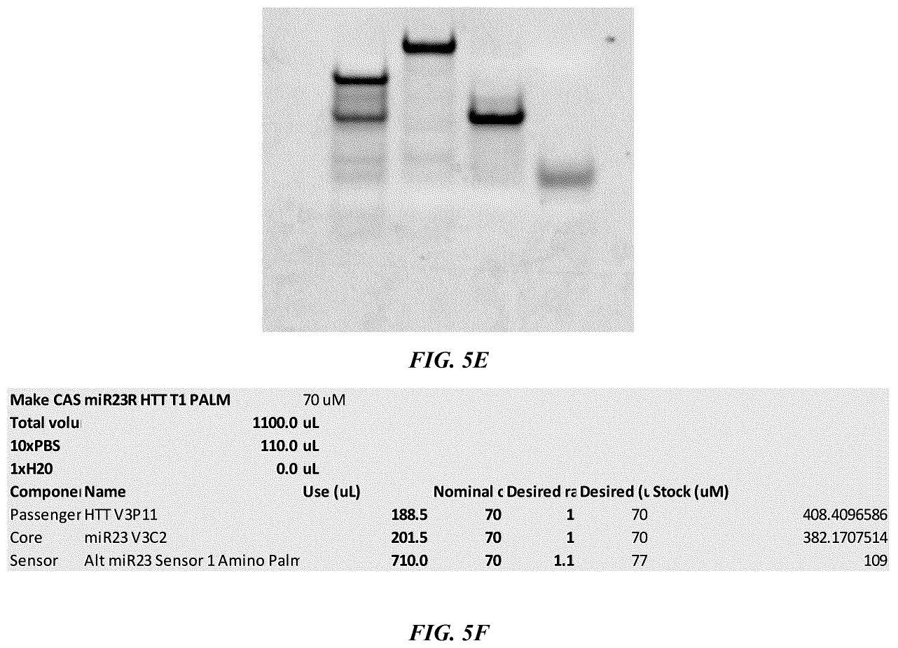

The T1 CASi consists of the sensor strand, core strand, and the passenger strand (FIG. 5A). The sensor strand is complementary to the guide strand of mir23a-3p, a microRNA with high expression in the brain tissue. The core strand has two domains. The first domain is complementary to the sensor strand to allow formation of the sensor duplex. The second domain is complementary to both rodent and primate Huntingtin gene mRNA. The passenger strand is complementary to the second domain of the core strand and base-pairs with the core strand to form the siRNA. A palmitic acid ligand can be added to the sensor strand to enhance delivery. In some T1 CASi constructs, the palmitic acid is attached to the 3′ end of the sensor strand.

Table 1 below provides sequence diagrams of exemplary TI CASi constructs and strands. The chemical formulas of the mir23 sensor strand (8 nt with palmitic acid), HTT passenger strand, and mir23-HTT core strands are shown in FIGS. 5B, 5C and 5D, respectively.

TABLE 1
Sequences of exemplary T1 CASi strands.

| | |
|---|---|
| HTT passenger | mU*mU*mA.mU.mA.mU.fC.mA.fG.fU.fA.mA.mA.mG.mA.mG.mA.mU.mU*mA*mA |
| strand | (SEQ ID NO: 1) |
| mir23-HTT core | mG.mC.mC.mA.mG.mG.mG.mA.mU.mU.mU.mC.mC.mG.mU*fU*mA.mA.mU.fC.mU. |
| strand | mC.mU.mU.mU.mA.mC.fU.mG.fA.mU.mA.mU.mA.mA*mU*mU |
| | (SEQ ID NO: 2) |
| mir23 sensor | mC* + G.mG.mA.mA.mA.mU.mC.mC.mC.mU.mG.mG.mC*mA*mA* + T*mG* + |
| (12 nt) | T*mG* + A*mU*mU*mU*mU*mU*/3AmMC6T/ |
| | (SEQ ID NO: 3) |
| mir23 sensor | mC* + G.mG.mA.mA.mA.mU.mC.mC.mC.mU.mG.mG.mC*mA*mA* + T*mG* + |
| (16 nt) | T*mG* + A*mU*mU*mU*mU*mA*mU*mU*mU*mA*/3AmMC6T/ |
| | (SEQ ID NO: 4) |
| mir23 sensor (8 | /5Sp9/*mC*mG* + G.mA. + A.mA.mU. + C.mC.mC. + T.mG* + G*mC*mA*mA* + |
| nt no palmitic | T*mG* + T*mG* + A* + T |
| acid) | (SEQ ID NO: 5) |
| mir23 sensor (8 | /5Sp9/*mC*mG* + G.mA. + A.mA.mU. + C.mC.mC. + T.mG* + G*mC*mA*mA* + |
| nt with palmitic | T*mG* + T*mG* + A* + T*/Palm/ |
| acid) | (SEQ ID NO: 6) |

/5Sp9/ = 5′ triethylene glycol spacer
/Palm/ = palmitic acid
* = phosphorothioate backbone modification
. = phosphodiester backbone
mA, mG, mC, mU = 2′-O-methyl bases
+A, +T, +C, +G = locked nucleic acid (LNA) bases
fA, fU, fC, fG = 2′-fluoro bases

The constructs were assembled by thermal annealing. Strands were mixed in PBS buffer at 1:1:1.1 ratio of passenger:core:sensor, then heated above 75° C., and then cooled to room temperature. Non-denaturing PAGE gel was used to compare the assembled construct (lane 2 in FIG. 5E) with the individual strands (lane 3 in FIG. 5E: core strand; lane 4 in FIG. 5E: passenger strand) and two-stranded sub-assemblies (lane 1 in FIG. 5E: RNAi duplex). Presence of a single band (lane 2) with slower migration than single strands (lanes 3 and 4) and duplexes (lane 1) indicates assembly of the correct construct.

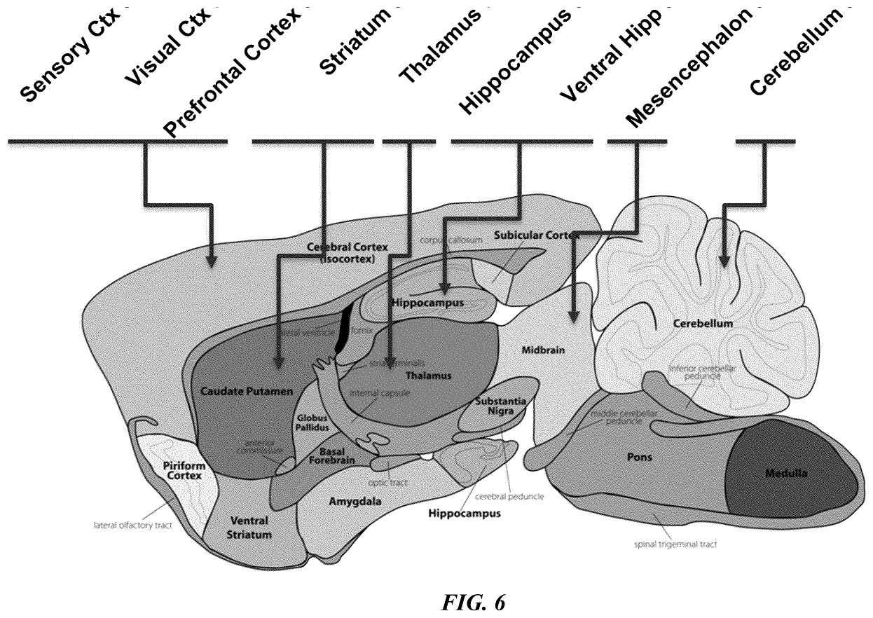

The assembled constructs were concentrated to ˜3 mM concentration in 1x PBS, then delivered by intracerebral ventricular injection (ICV) into the right ventricle of wild type mice. Mice were sacrificed at 14, 30, or 90 days after injection. The brain was collected and dissected into nine regions: sensory cortex, visual cortex, prefrontal cortex, striatum, thalamus, dorsal hippocampus (hippocampus), ventral hippocampus, mesencephalon (midbrain), and cerebellum (FIG. 6). Tissue from each brain region was lysed and assayed for HTT mRNA expression by qPCR. Knockdown of HTT mRNA was determined by comparing mRNA levels in construct-treated mice with saline-treated mice.

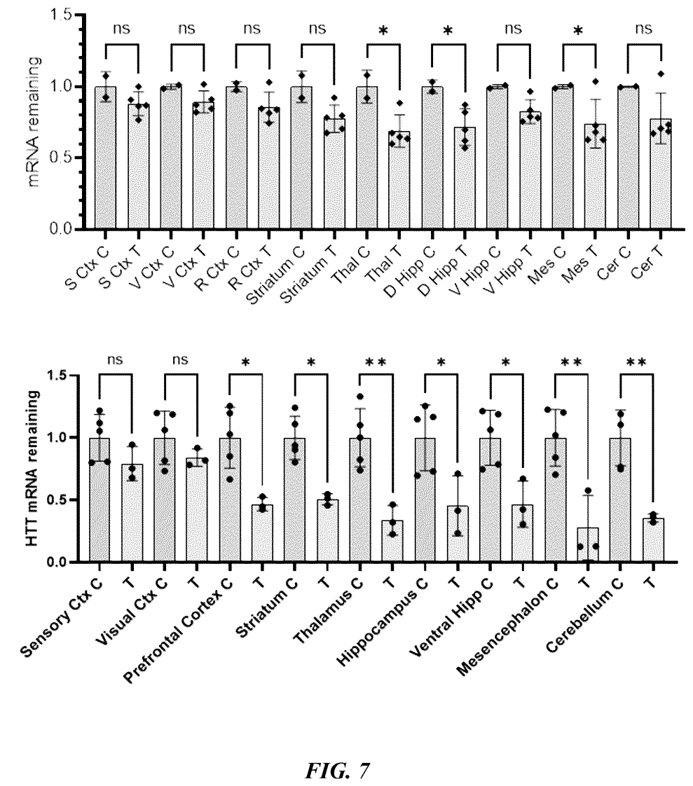

FIG. 7 shows mRNA knockdown of the mir23-HTT construct with a 3′ terminal palmitic acid (bottom panel) and without a 3′ terminal palmitic acid (top panel) in different brain regions 14 days after injection. The results demonstrate that mir23-HTT constructs with a 3′ terminal palmitic acid achieved a higher degree of HTT mRNA knockdown (lower amount of remaining HTT mRNA) across all brain regions compared to the mir23-HTT constructs without a 3′ terminal palmitic acid.

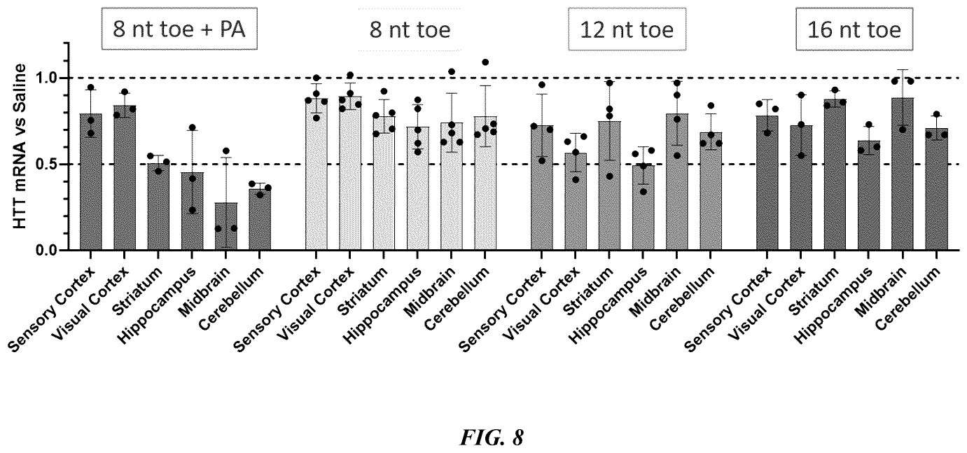

FIG. 8 depicts a diagram showing mRNA knockdown of mir23-HTT constructs having a standard 8 nucleotide toehold (with and without palmitic acid), an extended 12 nucleotide toehold, or 16 nucleotide toehold. The data suggests toehold length and palmitic acid have different effects on mir23-HTT CASi activity in different brain regions. CASi construct with a 8 nucleotide toehold and palmitic acid achieved overall best knockdown effects across the brain regions. Without the palmitic acid, increasing toehold length from 8 nt to 12 nt improved knockdown activity in cortex and hippocampus.

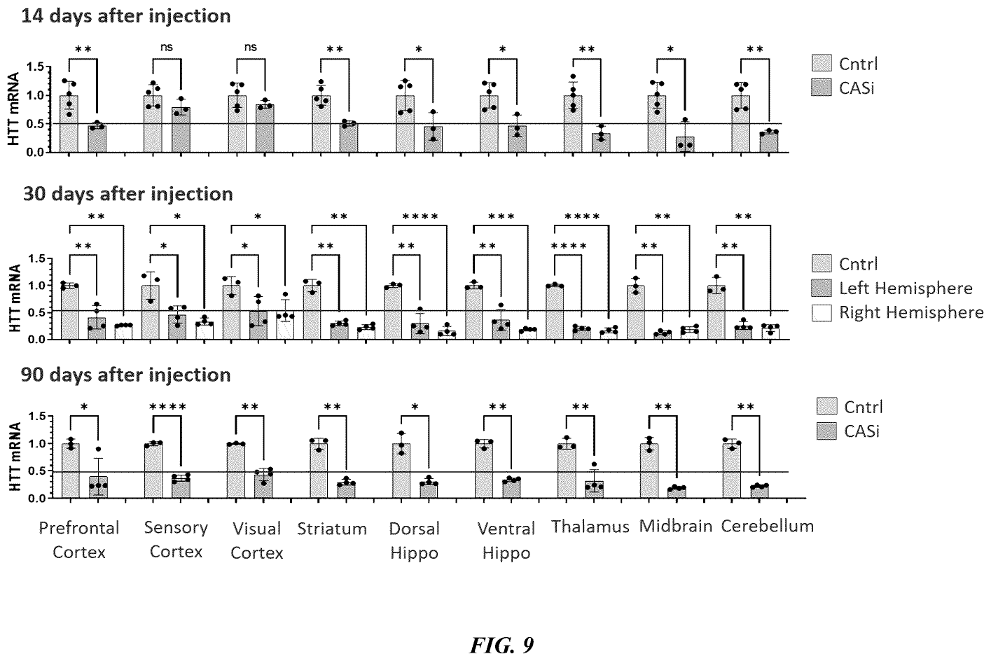

FIG. 9 shows mRNA knockdown of mir23-HTT construct (with a standard 8 nucleotide toehold and 3′ palmitic acid) at 14 days, 30 days, and 90 days after injection of a single 15 nmol dose (425 μg) by unilateral ICV in the right ventricle. The data suggests a potent and durable knockdown by the mir23-HTT CASi constructs in all brain regions. Statistically significant RNAi activity was observed in brain regions including prefrontal cortex, sensory cortex, visual cortex, striatum, dorsal hippocampus, ventral hippocampus, thalamus, midbrain, cerebellum, right hemisphere and left hemisphere regions of the brain. The data also demonstrates that the RNAi activity remains potent even 90 days after the injection, thus enabling a dosage regimen with less frequency of administration and longer time interval between administrations.

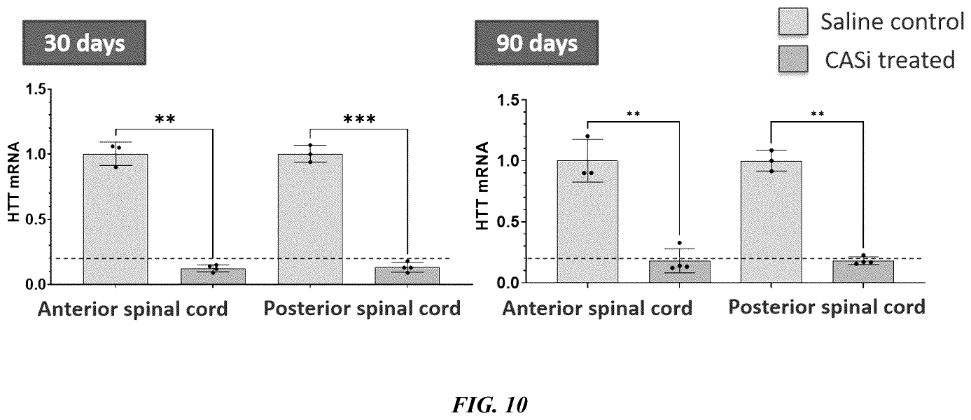

FIG. 10 shows HTT mRNA knockdown of mir23-HTT construct in the spinal cord. The data demonstrates that about 80% target knockdown rate still remains in the spinal cord 30 or even 90 days after the injection.

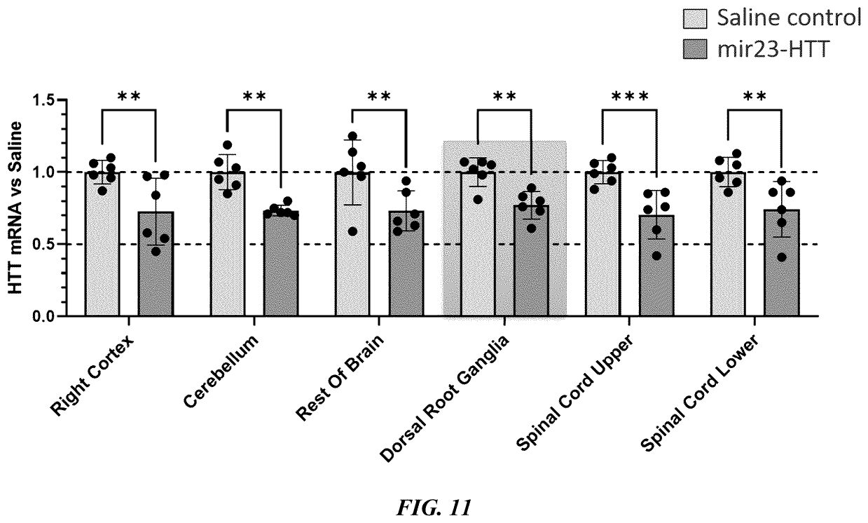

FIG. 11 showing the HTT mRNA level in various brain regions 30 days after CASi administration. 5 nM mir23-HTT CASi construct (8 nucleotide toehold, with palmitic acid) was administrated to the animals through a unilateral ICV injection. In addition to the brain regions and the spinal cord, 10 dorsal root ganglia (DRG) from mice were also collected and tested for HTT mRNA knockdown. About 25% HTT mRNA knockdown was observed across the central nervous system and DRG. The results suggest that not only was statistically significant RNAi activity observed in the central nervous system, the CASi construct also effectively reached the peripheral nervous region from central administration and achieved comparable knockdown in the DRG compared with the central nervous system.

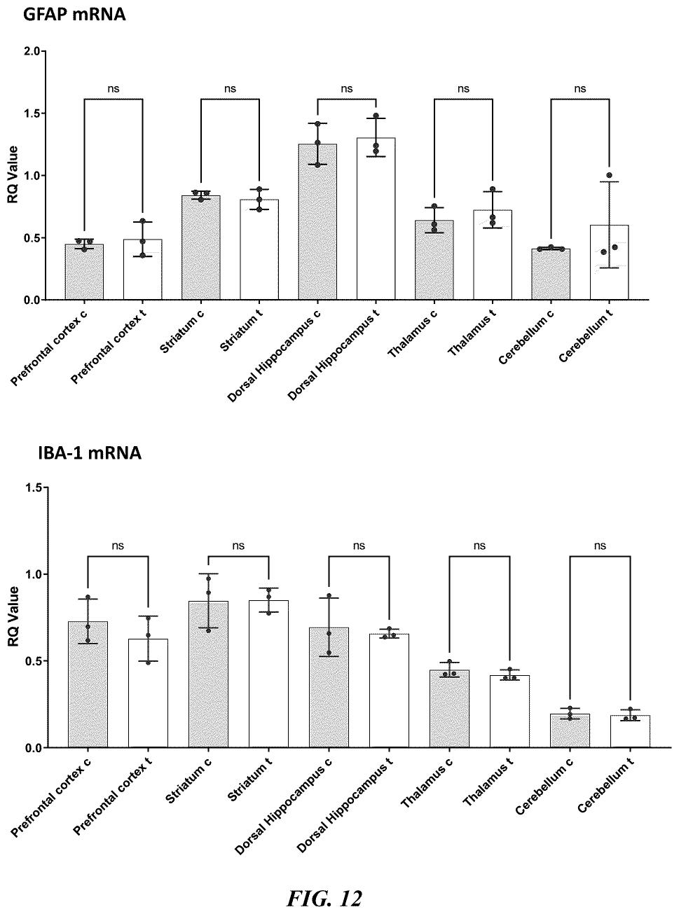

The mir23-HTT CASi construct (8 nucleotide toehold with palmitic acid) was also administered to mice via ICV injection at 15 nmol (425 μg) to test for CNS inflammation by measuring astrocyte (GFAP) and microglia (IBA-1) activation. FIG. 12 shows measurement of GFAP mRNA and IBA-1 mRNA by qPCR in various brain regions of CASi treated animals (t) in comparison to saline treated animals (c) at 14 days after injection. The mice treated with the CASi did not show elevated GFAP mRNA or IBA-1 mRNA compared with saline-treated mice, indicating that injection of the construct did not induce an unintended inflammatory response.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A nucleic acid complex, comprising:
a first nucleic acid strand comprising 20-60 linked nucleosides;
a second nucleic acid strand binding to a first region of the first nucleic acid strand to form a first nucleic acid duplex; and
a third nucleic acid strand binding to a second region of the first nucleic acid strand to form a second nucleic acid duplex, wherein the third nucleic acid strand comprises a overhang, wherein the overhang is not complementary to the first nucleic acid strand and is capable of binding to an input nucleic acid strand to cause the displacement of the third nucleic acid strand from the first nucleic acid strand,
wherein
the first region of the first nucleic acid strand is 3′ of the second region of the first nucleic acid strand,
the third nucleic acid strand does not bind to any region of the first nucleic acid strand that is 3′ of the first region of the first nucleic acid strand,
and the first region of the first nucleic acid strand comprises a sequence complementary to a target RNA, wherein the sequence is 10-35 nucleosides in length.

2. The nucleic acid complex of claim 1, wherein the sequence complementary to the target RNA is 10-21 nucleotides in length.

3. The nucleic acid complex of claim 1, wherein the second nucleic acid strand binds to 17-22 linked nucleotides in the first region of the first nucleic acid strand to form the first nucleic acid duplex, wherein the third nucleic acid strand binds to 10-30 linked nucleotides in the second region of the first nucleic acid strand to form the second nucleic acid duplex, or wherein the third nucleic acid strand binds to about 14 linked nucleotides in the second region of the first nucleic acid strand to form the second nucleic acid duplex.

4. (canceled)

5. (canceled)

6. The nucleic acid complex of claim 1, wherein the first nucleic acid duplex does not comprise a Dicer cleavage site or wherein the nucleic acid complex does not comprise a Dicer cleavage site.

7. (canceled)

8. The nucleic acid complex of claim 1, wherein the first region of the first nucleic acid strand is linked to the second region of the first nucleic acid strand via a linker, wherein the linker comprises a C₃ 3-carbon linker, a nucleotide, a modified nucleotide, or a exonuclease cleavage-resistant moiety, or a combination thereof, wherein the modified nucleotide is a 2′-O-methyl nucleotide or a 2′-F nucleotide.

9. (canceled)

10. (canceled)

11. The nucleic acid complex of claim 1, wherein the 5′ terminus of the second nucleic acid strand comprises a blocking moiety; wherein the blocking moiety comprises, or is, a fluorophore, an inverted-dT, a tri-ethylene-glycol, a fatty acid, a Cy3, or a combination thereof; and wherein the fluorophore is attached to the 5′ terminus of the second nucleic strand via a phosphorothioate linkage.

12. The nucleic acid complex of claim 1, wherein the first nucleic acid strand comprises a 3′ overhang that is one, two, or three nucleotides in length in the first nucleic acid duplex; and wherein the 3′ overhang of the first nucleic acid strand comprises one or more phosphorothioate internucleoside linkages or wherein all of the internucleoside linkages in the 3′ overhang of the first nucleic acid strand are phosphorothioate internucleoside linkages.

13. (canceled)

14. (canceled)

15. (canceled)

16. (canceled)

17. The nucleic acid complex of claim 1, wherein the first region of the first nucleic acid strand does not comprise phosphorothioate internucleoside linkages except for the internucleoside linkage(s) between the last two or three nucleosides at the 5′ terminus, 3′ terminus, or both; and
wherein the second region of the first nucleic acid strand does not comprise phosphorothioate internucleoside linkages.

18. (canceled)

19. (canceled)

20. The nucleic acid complex of claim 1, wherein the second nucleic strand is fully complementary to the first region of the first nucleic acid strand, thereby forming no overhang at the 5′ and 3′ termini of the second nucleic acid strand in the first nucleic acid duplex; and wherein the second nucleic acid strand comprises one or more phosphorothioate internucleoside linkages or wherein the second nucleic acid strand does not comprise phosphorothioate internucleoside linkages except for the internucleoside linkage(s) between the last two to three nucleosides at the 5′ terminus and the last two to three nucleosides at 3′ terminus.

21. (canceled)

22. (canceled)

23. (canceled)

24. (canceled)

25. The nucleic acid complex of claim 1, wherein the 5′ terminus of the third nucleic acid strand comprises at least one phosphorothioate internucleoside linkage; or wherein the last two, three or four nucleosides at the 5′ terminus of the third nucleic acid strand are phosphorothioate internucleoside linkages.

26. (canceled)

27. The nucleic acid complex of claim 1, wherein less than 5%, less than 10%, less than 25%, or less than 50% of the internucleoside linkages in the first nucleic acid strand are phosphorothioate internucleoside linkages; or
wherein the first nucleic acid strand comprises no more than two phosphorothioate internucleoside linkages, or does not comprise phosphorothioate internucleoside linkages.

28. (canceled)

29. (canceled)

30. The nucleic acid complex of claim 1, wherein at least 50%, at least 60%, at least 70%, at least 80%, at least 85%, at least 90%, at least 95%, or all of the nucleosides of one or more of the first nucleic acid strand, the second nucleic strand and the third nucleic strand are chemically modified; or wherein at least 50%, at least 60%, at least 70%, at least 80%, at least 85%, at least 90%, at least 95%, or all of the nucleosides of the nucleic acid complex are chemically modified.

31. (canceled)

32. (canceled)

33. (canceled)

34. (canceled)

35. (canceled)

36. (canceled)

37. (canceled)

38. (canceled)

39. (canceled)

40. The nucleic acid complex of claim 1, wherein the overhang of the third nucleic acid strand is capable of binding to the input nucleic acid strand to form a toehold, thereby causing the displacement of the third nucleic acid strand from the first nucleic acid strand; wherein the overhang of the third nucleic acid strand is 5 to 20 nucleosides in length; and wherein all internucleoside linkages of the overhang of the third nucleic acid strand are phosphorothioate internucleoside linkages.

41. (canceled)

42. (canceled)

43. The nucleic acid complex of claim 1, wherein the 5′ terminus, the 3′ terminus, or both of the third nucleic acid strand comprises a terminal moiety comprising a ligand, a fluorophore, a exonuclease, a fatty acid, a Cy3, an inverted dT attached to a tri-ethylene glycol, or a combination thereof.

44. (canceled)

45. The nucleic acid complex of claim 43, wherein the terminal moiety comprises a palmitic acid attached to the 3′ terminus of the third nucleic acid strand.

46. A method of modulating a target RNA, comprising:
contacting a cell comprising a target RNA with a nucleic acid complex comprising:
a first nucleic acid strand comprising 20-60 linked nucleosides;
a second nucleic acid strand binding to a first region of the first nucleic acid strand to form a first nucleic acid duplex; and
a third nucleic acid strand binding to a second region of the first nucleic acid strand to form a second nucleic acid duplex, wherein the third nucleic acid strand comprises a overhang, wherein the overhang is not complementary to the first nucleic acid strand and is capable of binding to an input nucleic acid strand to cause the displacement of the third nucleic acid strand from the first nucleic acid strand,
wherein the first region of the first nucleic acid strand is 3′ of the second region of the first nucleic acid strand,
the third nucleic acid strand does not bind to any region of the first nucleic acid strand that is 3′ of the first region of the first nucleic acid strand,
and the first region of the first nucleic acid strand comprises a sequence complementary to a target RNA, wherein the sequence is 10-35 nucleosides in length; and
wherein the input strand binds to the overhang of the third nucleic acid strand to cause displacement of the third nucleic acid strand from the first nucleic acid strand to release the sequence complementary to the target RNA into the cell, thereby modulating the target RNA.

47. The method of claim 46, wherein contacting the cell with the nucleic acid complex is performed in vitro, in vivo, ex vivo, or a combination thereof; and wherein the cell is a cancer cell or a neuron.

48. (canceled)

49. (canceled)

50. (canceled)

51. A method of treating a disease or a condition, comprising administering a nucleic acid complex to a subject in need thereof, wherein the nucleic acid complex comprises:
a first nucleic acid strand comprising 20-60 linked nucleosides;
a second nucleic acid strand binding to a first region of the first nucleic acid strand to form a first nucleic acid duplex; and
a third nucleic acid strand binding to a second region of the first nucleic acid strand to form a second nucleic acid duplex, wherein the third nucleic acid strand comprises a overhang, wherein the overhang is not complementary to the first nucleic acid strand and is capable of binding to an input nucleic acid strand to cause the displacement of the third nucleic acid strand from the first nucleic acid strand,
wherein the first region of the first nucleic acid strand is 3′ of the second region of the first nucleic acid strand,
the third nucleic acid strand does not bind to any region of the first nucleic acid strand that is 3′ of the first region of the first nucleic acid strand,
and the first region of the first nucleic acid strand comprises a sequence complementary to a target RNA, wherein the sequence is 10-35 nucleosides in length; and
wherein the input strand binds to the overhang of the third nucleic acid strand to cause displacement of the third nucleic acid strand from the first nucleic acid strand to release the sequence complementary to a target RNA, thereby reducing the activity of the target RNA or protein expression from the target RNA in the subject to treat the disease or condition.

52. (canceled)

53. (canceled)

54. The method of claim 51, wherein the disease or condition is a neurological disease and wherein the administration results in the distribution of the nucleic acid complex into one or more regions selected from the group consisting of: right cortex, prefrontal cortex, sensory cortex, visual cortex, striatum, dorsal hippocampus, ventral hippocampus, thalamus, cerebellum, midbrain, left hemisphere, right hemisphere, dorsal root ganglia, or a combination thereof or wherein the administration results in the distribution of the nucleic acid complex into dorsal root ganglia.

55. (canceled)

56. (canceled)

57. The method of claim 51, wherein the nucleic acid complex is administered to a subject via a lipid-mediated delivery system comprising liposomes, nanoparticles, or micelles; and
wherein the nucleic acid complex is administered to the subject in need thereof via a subcutaneous injection, an intravenous injection, or an intracerebroventricular injection; and wherein the nucleic acid complex is administered to the subject in need thereof at a concentration about 0.1-10 nM.

58. (canceled)

59. (canceled)

60. (canceled)

61. (canceled)

62. (canceled)


SEQUENCE LISTING (12 sequences)

SEQ ID NO: 1 - RNA, 21 nt, synthetic construct
  source 1..21: mol_type = other RNA
  modified_base 1..2: mod_base = um
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = um
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroadenosine
  modified_base 12..13: mod_base = OTHER; note = 2'-O-methyladenosine
  modified_base 14: mod_base = gm
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18..19: mod_base = um
  modified_base 20..21: mod_base = OTHER; note = 2'-O-methyladenosine
  modified_base 1..3: mod_base = OTHER; note = phosphorothioate backbone
  modified_base 19..21: mod_base = OTHER; note = phosphorothioate backbone
ttatatcagtaaagagattaa

SEQ ID NO: 2 - RNA, 37 nt, synthetic construct
  source 1..37: mol_type = other RNA
  modified_base 1..15: mod_base = OTHER; note = 2'-O-methyl base
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = gm
  modified_base 30: mod_base = OTHER; note = 2-fluoro base
  modified_base 31..37: mod_base = OTHER; note = 2'-O-methyl base
  modified_base 15..17: mod_base = OTHER; note = phosphorothioate backbone
  modified_base 35..37: mod_base = OTHER; note = phosphorothioate backbone
gccagggatttccgttaatctctttactgatataatt

SEQ ID NO: 3 - RNA, 26 nt, synthetic construct
  source 1..26: mol_type = other RNA
  modified_base 1: mod_base = cm
  modified_base 2: mod_base = OTHER; note = locked nucleic acid base
  modified_base 3..16: mod_base = OTHER; note = 2'-O-methyl base
  modified_base 17: mod_base = OTHER; note = locked nucleic acid base
  modified_base 18: mod_base = gm
  modified_base 19: mod_base = OTHER; note = locked nucleic acid base
  modified_base 20: mod_base = gm
  modified_base 21: mod_base = OTHER; note = locked nucleic acid base
  modified_base 22..26: mod_base = OTHER; note = 2'-O-methyl base
  modified_base 1^2: mod_base = OTHER; note = phosphorothioate bond
  modified_base 14..26: mod_base = OTHER; note = phosphorothioate backbone
  misc_binding 26: bound_moiety = 3' Amino Modifier C6 dT
cggaaatccctggcaatgtgattttt

SEQ ID NO: 4 - RNA, 30 nt, synthetic construct
  source 1..30: mol_type = other RNA
  modified_base 1: mod_base = cm
  modified_base 2: mod_base = OTHER; note = locked nucleic acid base
  modified_base 3..16: mod_base = OTHER; note = 2'-O-methyl base
  modified_base 17: mod_base = OTHER; note = locked nucleic acid base T
  modified_base 18: mod_base = gm
  modified_base 19: mod_base = OTHER; note = locked nucleic acid base T
  modified_base 20: mod_base = gm
  modified_base 21: mod_base = OTHER; note = locked nucleic acid base
  modified_base 22..30: mod_base = OTHER; note = 2'-O-methyl base
  modified_base 1^2: mod_base = OTHER; note = phosphorothioate bond
  modified_base 14..30: mod_base = OTHER; note = phosphorothioate backbone
  misc_binding 30: bound_moiety = 3' amino modifier C6 dT
cggaaatccctggcaatgtgattttattta

SEQ ID NO: 5 - RNA, 22 nt, synthetic construct
  source 1..22: mol_type = other RNA
  modified_base 1: mod_base = cm
  modified_base 2: mod_base = gm
  modified_base 3: mod_base = OTHER; note = locked nucleic acid base
  modified_base 4: mod_base = OTHER; note = 2'-O-methyl base
  modified_base 5: mod_base = OTHER; note = locked nucleic acid base
  modified_base 6..7: mod_base = OTHER; note = 2'-O-methyl base
  modified_base 8: mod_base = OTHER; note = locked nucleic acid base
  modified_base 9..10: mod_base = cm
  modified_base 11: mod_base = OTHER; note = locked nucleic acid base T
  modified_base 12: mod_base = gm
  modified_base 13: mod_base = OTHER; note = locked nucleic acid base
  modified_base 14..16: mod_base = OTHER; note = 2'-O-methyl base
  modified_base 17: mod_base = OTHER; note = locked nucleic acid base T
  modified_base 18: mod_base = gm
  modified_base 19: mod_base = OTHER; note = locked nucleic acid base T
  modified_base 20: mod_base = gm
  modified_base 21: mod_base = OTHER; note = locked nucleic acid base
  modified_base 22: mod_base = OTHER; note = locked nucleic acid base T
  modified_base 1..3: mod_base = OTHER; note = phosphorothioate backbone
  modified_base 12..22: mod_base = OTHER; note = phosphorothioate backbone
  misc_binding 1: bound_moiety = 5' triethylene glycol spacer
cggaaatccctggcaatgtgat

SEQ ID NO: 6 - RNA, 22 nt, synthetic construct
  source 1..22: mol_type = other RNA
  modified_base 1: mod_base = cm
  modified_base 2: mod_base = gm
  modified_base 3: mod_base = OTHER; note = locked nucleic acid base
  modified_base 4: mod_base = OTHER; note = 2'-O-methyl base
  modified_base 5: mod_base = OTHER; note = locked nucleic acid base
  modified_base 6..7: mod_base = OTHER; note = 2'-O-methyl base
  modified_base 8: mod_base = OTHER; note = locked nucleic acid base
  modified_base 9..10: mod_base = cm
  modified_base 11: mod_base = OTHER; note = locked nucleic acid base T
  modified_base 12: mod_base = gm
  modified_base 13: mod_base = OTHER; note = locked nucleic acid base
  modified_base 14..16: mod_base = OTHER; note = 2'-O-methyl base
  modified_base 14: mod_base = OTHER; note = locked nucleic acid base T
  modified_base 18: mod_base = gm
  modified_base 17: mod_base = OTHER; note = locked nucleic acid base T
  modified_base 19: mod_base = OTHER; note = locked nucleic acid base T
  modified_base 20: mod_base = gm
  modified_base 21: mod_base = OTHER; note = locked nucleic acid base
  modified_base 22: mod_base = OTHER; note = locked nucleic acid base T
  modified_base 1..3: mod_base = OTHER; note = phosphorothioate backbone
  modified_base 12..22: mod_base = OTHER; note = phosphorothioate backbone
  misc_binding 1: bound_moiety = 5' triethylene glycol spacer
  misc_binding 22: bound_moiety = palmitic acid
cggaaatccctggcaatgtgat

SEQ ID NO: 7 - RNA, 21 nt, synthetic construct
  source 1..21: mol_type = other RNA
  misc_binding 1: bound_moiety = Cy3
  modified_base 1: mod_base = OTHER; note = 2'-O-methyl U
  modified_base 2: mod_base = OTHER; note = locked nucleic acid A
  modified_base 3: mod_base = cm
  modified_base 4: mod_base = OTHER; note = 2'-O-methyladenosine
  modified_base 5..6: mod_base = gm
  modified_base 7: mod_base = OTHER; note = 2'-fluoro A
  modified_base 8: mod_base = OTHER; note = 2'-O-methyladenosine
  modified_base 9..11: mod_base = OTHER; note = 2'-fluoro base
  modified_base 12..21: mod_base = OTHER; note = 2'-O-methyl base
  modified_base 1..3: mod_base = OTHER; note = phosphorothioate backbone
  modified_base 19..21: mod_base = OTHER; note = phosphorothioate backbone
tacaggaaaagccaaacaaca

SEQ ID NO: 8 - RNA, 24 nt, synthetic construct
  source 1..24: mol_type = other RNA
  misc_feature 1: note = The 5 terminus is attached to a spacer 9 via phosphorothioate backbone.
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  modified_base 1..3: mod_base = OTHER; note = phosphorothioate backbone
  modified_base 12..24: mod_base = OTHER; note = phosphorothioate backbone
gaccaagctgcgtgacacaccact

SEQ ID NO: 9 - RNA, 37 nt, synthetic construct
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 1..3: mod_base = OTHER; note = phosphorothioate backbone
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base
cacgcagcttggtctgttgtttggcttttcctgtaca

SEQ ID NO: 10 - RNA, 24 nt, synthetic construct
  source 1..24: mol_type = other RNA
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  modified_base 1..3: mod_base = OTHER; note = phosphorothioate backbone
  modified_base 12..24: mod_base = OTHER; note = phosphorothioate backbone
gaccaagctgcgtgacacaccact

SEQ ID NO: 11 - RNA, 37 nt, synthetic construct; identical to SEQ ID NO: 9
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base

SEQ ID NO: 12 - RNA, 37 nt, synthetic construct; identical to SEQ ID NO: 9
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base
  misc_feature 1: note = 5' spacer 9